(19) United States
(12) Patent Application Publication
(10) Pub. No.: US 2015/0159170 A1
(43) Pub. Date: Jun. 11, 2015

(54) NOVEL PLANT-DERIVED CIS-REGULATORY ELEMENTS FOR THE DEVELOPMENT OF PATHOGEN-RESPONSIVE CHIMERIC PROMOTORS

(71) Applicant: KWS SAAT AG, Einbeck (DE)

(72) Inventors: Dietmar Stahl, Einbeck (DE); Stephanie Meinecke, Einbeck (DE)

(73) Assignee: KWS SAAT AG, Einbeck (DE)

(21) Appl. No.: 14/380,545

(22) PCT Filed: Feb. 26, 2013
(86) PCT No.: PCT/DE2013/000098

§ 371 Date: Aug. 22, 2014

(30) Foreign Application Priority Data
Feb. 29, 2012 (DE) 10 2012 003 848.8

(51) Int. Cl. C12N 15/82

(52) U.S. Cl. CPC C12N 15/8279; C12N 15/8282; C12N 15/8239

(57) ABSTRACT

The present invention provides pathogen-resistant transgenic plants which exhibit a resistance protein-mediated pathogen defence reaction in a cell of the plant under stringent control owing to a pathogen infection. In this case, as inducer of the pathogen defence, parts of avirulence proteins are used, the stable integration of which is possible by means of usual transformation methods. In addition, the invention relates to a composition of nucleic acids which, after integration into the genome of a plant, mediates the pathogen resistance therein, to a method for producing a pathogen-resistant plant, and to plants for producing a pathogen-resistant plant.

The present invention relates to a pathogen-resistant plant, in particular a plant having a new type of resistance based on the reaction of a number of parts of an avirulence protein with a corresponding resistance protein in a cell of the plant, a composition of nucleic acids, which, following integration into the genome of a plant, conveys the pathogen resistance in said plant, a method for producing a pathogen-resistant plant, and plants for producing a pathogen-resistant plant.

Plant diseases caused by phytopathogens, such as fungi, viruses, nematodes and bacteria, cause large harvest losses globally, considerably impair the quality of the harvest products, and make necessary a costly use of chemical plant protection agents. The natural measures of the plant immune system with the aid of which it is possible to defend against the majority of potential pathogens or to delay and limit the spread of said pathogens are often inadequate.

The plant immune system reacts to a pathogen attack. In a first phase, transmembrane receptors (PRRs, pattern recognition receptors) identify molecular patterns of a pathogen (what are known as MAMPs or PAMPs, microbial- or pathogen-associated molecular patterns) and provide the plant with “PAMP-triggered immunity” (PTI), which is intended to prevent a further spread of the pathogen.

However, pathogens have developed strategies for surviving this first defence reaction. Pathogens use a wide range of different infection pathways: Whereas pathogenic bacteria for example can infiltrate the plant via stomata and hydathodes or as a result of a wound and multiply there in the apoplastic space, fungi infiltrate directly into the epidermal plant cells or form hyphae on or between the epidermal cells, by means of which they are also able, once they have reached the stomata, to grow into the plant tissue via the stomata. In spite of all the differences, a feature common to all pathogen classes however is that, during the course of the infection, they release effector molecules (virulence factors) into the plant cells, where these influence the virulence of the pathogen significantly. Some effectors are thus able to weaken the PTI in such a way that successful colonisation of the host plant is made possible (effector triggered susceptibility, ETS) (Jones & Dangl, 2006).

Further phases of the plant immune defence are directed against this ETS. Some effector molecules (the avirulence proteins) planted into the plant cell are identified very specifically by plant NBS-LRR resistance proteins (R proteins). Here, the avirulence proteins react either directly or indirectly with the corresponding R protein in accordance with the guard hypothesis, whereupon the R protein is activated (Dangl & Jones, 2001; Jones & Dangl, 2006). The activated R protein is able to trigger a signal cascade, which causes an accelerated and amplified PTI in the plant or what is known as effector triggered immunity, ETI (Jones & Dangl, 2006). Avirulence proteins thus generally constitute inducers of a plant pathogen defence reaction. This expresses itself in different physiological reactions of the plant, as in a hypersensitive reaction (HR), a further reinforcement of the cell wall by lignification and callose formation, in the synthesis of phytoalexins, the production of PR (pathogenesis-related) proteins, and often also in the controlled cell death of the host tissue at the site of infection of the pathogen.

In spite of these measures of the plant immune system, some pathogens are still able to impede ETI and successfully infect the plant, for example by diversifying the identified effectors or providing additional ETI-inhibiting effectors. One objective of cultivation and research has therefore already long been that of further increasing the resistance of plants, preferably of crops, to pathogens, whereby it is intended in particular to improve the plants in such a way that they are made resistant to a multiplicity of pathogens at the same time (broad pathogen resistance).

As early as the start of the 1990s, de Wit presented his visionary concept with this objective in WO/1991/15585 (also in de Wit, 1992): It is based on the pathogen-induced co-expression of a plant resistance gene and of the corresponding avirulence gene from the pathogen in a cell of a plant, whereby, following pathogen attack, a defence reaction of the plant limited to the site of the infection is to be induced by activation of the synthesised resistance protein, moreover more quickly and more effectively than would occur naturally as a result of the measures of the plant immune system.

However, it has long been considered that this concept is not feasible. Thus, reference is already made in WO/1995/31564 to the fact that in particular it is not clear from WO/1991/15585 which polynucleotide sequences can be used as suitable promoters for a broad pathogen resistance. This thus also means that, in the case of the proposed tomato resistance gene Cf-9 in combination with the avirulence gene Avr9 from Cladosporium fulvum, the induced necrosis due to the specificity of the proposed promoters could lead to a continued induction of Cf-9 and/or Avr9, which could result in uncontrolled necrosis as a result of the hypersensitive reaction.

Only in 1999 was it reported for the first time by Joosten and de Wit, that it was supposedly possible to transform a Cf-9-carrying tomato plant using an Avr9 gene under the transcriptional control of a shortened prp1-1 (gst1) promoter (Martini et al., 1993) and to thus produce an increased fungi resistance of tomatoes to a number of fungi (Joosten & de Wit, 1999). The two authors, however, also concede that there is a further need for optimisation in terms of the stringent regulation of the promoter and an unwanted induction of the gst1 promoter fragment in uninfected tissue (Strittmatter et al., 1996).

WO/1999/43823 discloses the production of transgenic maize plants, which are biolistically transformed with the fungal avirulence gene avrRxv under the control of a pathogen-inducible promoter, whilst they already naturally contained the corresponding resistance protein. The used pathogen-induced promoters, however, are not specific enough for an approach for obtaining broad pathogen resistance due to a high background activity. In addition, some of the proposed promoters in some circumstances trigger a systemic response to a pathogen infection, which would lead to an unwanted activation of these promoters even in uninfected cells. The fact that the technical teaching from WO/1999/43823 can be applied at best only to avirulence genes with a weak pathogen defence induction is also presented in the proposal of the authors to also use, alternatively to the pathogen-inducible promoters, weak constitutive promoters for expression control of the avirulence gene.

Although the prior art already provides methods for producing plants and produced plants with an improved pathogen resistance in accordance with the concept of de Wit, this technical teaching of the above prior art, however, cannot be readily transferred and applied to the integration of any avirulence genes, in particular those that code for strong inducers of a cell death trigger. The reason for this does not lie directly in the insufficiently stringent regulation of the transgene expression of the avirulence gene, but rather in the difficulty of integrating such an avirulence gene in a stable manner into the genome of a plant under the control of a pathogen-inducible promoter. This is because the pathogen-inducible plant promoters known today are already induced in an unwanted manner during the implementation of the usual techniques of plant transformation, which are also proposed from the prior art for stable genomic integration of an avirulence gene. This concerns both Agrobacterium tumefaciens-conveyed and biolistic transformation processes. By way of example, plants, in particular those that are less susceptible to transformation processes, are thus able to react via MAMP- or PAMP-responsive receptors in the plant cell membrane to the presence of A. tumefaciens directly or indirectly with the activation of pathogen-inducible promoters (Jones & Dangl, 2006; Kuta & Tripathi, 2005; WO/2007/068935). Numerous pathogen-inducible promoters are also activated by use, as occur for example during biolistic transformation (Stahl et al., 2006). The result is in any case the unwanted expression of the introduced avirulence gene. The synthesised avirulence protein reacts with the corresponding resistance protein already present and triggers the plant defence measures (ETI). This therefore already generally leads to the fact that either the vitality of these transformed cells is already severely limited, even without “real pathogen infection”, or the transformed cells are even destroyed in a controlled manner. This circumstance has, until now, completely prevented in particular the transformation of avirulence genes that code for inducers of cell death-triggering HR reactions in the presence of a corresponding resistance protein and the successful regeneration of the transformed cells into vigorous plants.

The invention has been developed against the background of the above-described prior art, wherein the object of the present invention was to provide a transgenic pathogen-resistant plant, in which a stringently regulated resistance protein-conveyed plant defence reaction with cell death trigger takes place by means of a stable integration of a pathogenic inducer as a result of a pathogen infection.

In accordance with the invention, the stated object is achieved by a pathogen-resistant plant, comprising at least two nucleic acids integrated in a stable manner into the genome, wherein the nucleic acids

(i) code for different parts of an avirulence protein and
(ii) are operatively linked to promoters,
and at least one of the promoters is pathogen-inducible, such that the different parts of the avirulence protein are present in synthesised form in a cell of the plant as a result of an infection of the plant by the pathogen and react directly or indirectly with a corresponding resistance protein.

Some of the terms used in this application will first be explained in greater detail below:

An “avirulence protein” is coded by an “avirulence gene” and constitutes an effector molecule of a pathogen, which plays a key role in the case of pathogen identification by the plant immune defence. In conjunction with the plant pathogen defence, an avirulence protein is characterised functionally in that it is able to react directly or indirectly with a corresponding resistance protein, provided this is present, in a plant cell, which then leads to the triggering of a plant pathogen defence reaction. Physiological reactions of the plant thus attained include, for example, a hypersensitive reaction (HR), a further reinforcement of the cell wall by lignification and callose formation, the synthesis of phytoalexins, the production of PR (pathogenesis-related) proteins and preferably also the controlled cell death of the host tissue, in particular at the site of pathogen infection.

Avirulence proteins can differ in respect of the degree of their induction capability for a cell death-triggering HR reaction in the presence of a corresponding resistance protein. Whether an avirulence protein functions as a strong or weak inducer in a plant cell is based substantially on the efficacy of the corresponding resistance protein in the plant species into which the avirulence gene/protein is introduced. If the resistance gene coding the resistance protein was introduced by means of gene-technology methods into a plant that does not naturally contain this resistance gene, further factors such as a position effect of a certain integration site or the inducibility of the promoter linked with the resistance gene can also have effects on the efficacy of a resistance protein and therefore also on the induction power of an avirulence protein in this plant.

The term “hybridise” used here means to hybridise under conventional conditions, as described in Sambrook et al. (1989), preferably under stringent conditions. Stringent hybridisation conditions are, for example: Hybridise in 4×SSC at 65° C. and then multiple washing in 0.1×SSC at 65° C. for a total of approximately 1 hour. The term “stringent hybridisation conditions” used here can also mean: Hybridise at 68° C. in 0.25 M sodium phosphate, pH 7.2, 7% SDS, 1 mM EDTA and 1% BSA for 16 hours and subsequent washing twice with 2×SSC and 0.1% SDS at 68° C.

The term “infection” is to be understood to mean the earliest moment at which the metabolism of a pathogen is prepared for a penetration of the host tissue. This includes, for example in the case of fungi, the growth of hyphae or the formation of specific infection structures, such as penetration hyphae and appressoria. A “true pathogen infection” comprises any infection of a plant with a pathogen or any use as a result of which a pathogen infection can take place. However, pathogen infections and uses of plants and plant cells that occur deliberately and selectively during the course of a gene technology-based method, such as Agrobacterium tumefaciens-conveyed transformation or biolistic transformation, are excluded.

“Complementary” nucleotide sequence means based on a nucleic acid in the form of a double-strand

DNA and that the second DNA strand complementary to the first DNA strand, in accordance with the base pair rules, has the nucleotides that correspond to the bases of the first strand.

Plant “organs” for example mean leaves, stems, trunk, roots, vegetative buds, meristems, embryos, anthers, ovula or fruits. Plant “parts” mean a combination of a number of organs, for example a flower or a seed, or part of an organ, for example a cross section through the stem. Plant “tissues” for example are callus tissue, storage tissue, meristematic tissue, leaf tissue, stem tissue, root tissue, plant tumour tissue or reproductive tissue. Plant “cells” for example are to be understood to mean isolated cells with a cell wall or aggregates thereof or protoplasts.

A “pathogen” in conjunction with the invention means organisms that, in interaction with a plant, lead to disease symptoms at one or more organs in the plant. These pathogens include animal, fungal, bacterial and viral organisms. Animal pathogens in particular comprise those of the roundworm strain (nematodes), such as species of the genera Anguina, Ditylenchus, Globodera, Heterodera, Meloidogyne, Paratrichodorus, Pratylenchus and Trichodorus, and those of the class of insects (Insecta), such as species of the genera Agriotes, Aphis, Atomaria, Autographa, Blithophaga, Cassida, Chaetocnema, Cleonus, Lixus, Lygus, Mamestra, Mycus, Onychiurus, Pemphigus, Philaenus, Scrobipalpa and Tipula. The fungal pathogens for example are selected from the divisions Plasmodiophoromycota, Oomycota, Ascomycota, Basidiomycota or Deuteromycota, which include, for example, species of the genera Actinomycetes, Alternaria, Aphanomyces, Botrytis, Cercospora, Erysiphe, Fusarium, Helicobasidium, Peronospora, Phoma, Phytium, Phytophthora, Pleospora, Ramularia, Rhizoctonia, Typhula, Uromyces and Verticillium. Bacterial pathogens include, for example, species of the genera Agrobacterium, Erwinia, Pseudomonas, Streptomyces and Xanthomonas, and viral pathogens include, for example, species of the genera Benyvirus, Closterovirus, Curtovirus, Luteovirus, Nucleorhabdovirus, Potyvirus and Tobravirus.

A “promoter” is a non-translated DNA portion, typically upstream of a coding region, which contains the binding point for the RNA polymerase and initiates the transcription of the DNA. A promoter additionally contains other elements that act as regulators of gene expression (for example cis-regulatory elements).

A “core or minimal promoter” is a promoter that has at least the basic elements used for transcription initiation (for example TATA box and/or initiator).

Here, a “synthetic promoter” or “chimeric promoter” is a promoter that does not occur in nature, is composed from a number of elements and contains a core or minimal promoter and also has, upstream of the core or minimal promoter, at least one cis-regulatory element, which serves as a binding point for special trans-acting factors (for example transcription factors). A synthetic or chimeric promoter is designed in accordance with the desired requirements and is induced or repressed by different factors. The selection of the cis-regulatory element or a combination of cis-regulatory elements is key for the specificity and the activity level of a promoter. In a synthetic or chimeric promoter, a core or minimal promoter can be functionally associated with one or more cis-regulatory elements, wherein the promoter/cis-element combination(s) are not known from natural promoters or are formed differently from natural promoters. Examples are known from the prior art (WO/00/29592; WO/2007/147395). A “pathogen-inducible promoter” is a promoter that is able to express the gene that it regulates following pathogen identification and/or a pathogen infection and/or a use, which may also be the result of an abiotic influence.

“Transgenic plant” refers to a plant in the genome of which at least one heterologous nucleic acid (for example an avirulence gene or also a fragment of an avirulence gene from a bacterial pathogen) has been integrated in a stable manner, which means that the integrated nucleic acid remains in the plant in a stable manner, is expressed and can also be inherited by the descendants in a stable manner. The stable integration of a nucleic acid in the genome of a plant also includes the integration in the genome of a plant of the previous parental generation, wherein the integrated nucleic acid can be passed on in a stable manner.

A pathogen-resistant plant according to the invention has at least two nucleic acids integrated in a stable manner in the genome. Each of these nucleic acids is characterised by a nucleotide sequence, which in each case codes for a different part of an avirulence protein. The nucleic acids constitute different fragments of the same avirulence gene. Each nucleic acid comprises at least one fragment of the avirulence gene.

Two or more nucleic acids may have nucleotide sequences that code for two or more different parts of the avirulence protein with amino acid sequences that are identical or similar in portions. The term “in portions” means that no amino acid sequence is present over its entire length in a manner 100% identical or similar in another amino acid sequence. Identical amino acid sequences are those of which the amino acid sequences correspond to one another, similar amino acid sequences display one or more conservative and/or semi-conservative amino acid substitutions based on similar physio-chemical properties of the different amino acids Amino acid sequences that are identical or similar in portions can also be terminally overlapping, such that, for example, a sequence at the C-terminus has an identical or similar sequence with a different sequence at the N-terminus Such amino acid sequences preferably overlap over a length of more than 3 successive amino acids, particularly preferably over a length of at least 11 successive amino acids. Similar overlapping amino acid sequences have a similarity of 60%, 70%, 80%, 90%, 95%, 96%, 97%, 98% or 99% in the overlapping sequence region, whereas identical overlapping amino acid sequences match in the overlapping sequence region. The match can be determined in accordance with known methods, for example computer-assisted sequence comparisons (Altschul et al., 1990).

A nucleic acid can also be further modified by addition, substitution or deletion of one or more nucleotides. For example, a nucleic acid can be provided with a start codon ATG (translation start) and/or stop codon in order to ensure a stable translation of the nucleic acid in a plant cell, or intron sequences can be deleted. Modifications of this type and implementation thereof are known to a person skilled in the art. Modified nucleic acids also include nucleic acids that hybridise under usual conditions (Sambrook et al. 1989), preferably under stringent conditions, with the corresponding non-modified nucleic acid or at DNA level demonstrate a homology of at least 60%, 70%, 80%, 90%, 95%, 96%, 97%, 98% or 99% to the non-modified nucleic acid. The amino acid sequence of an avirulence protein part coded by a modified nucleic acid may have an identity of at least 60%, 70%, 80%, 90%, 95%, 96%, 97%, 98% or 99% or a similarity of at least 60%, 70%, 80%, 90%, 95%, 96%, 97%, 98% or 99% with the original amino acid sequence.

An individual fragment of the avirulence gene codes for a non-functional part of the avirulence protein. This means that, rather than the entire avirulence protein, an individual part of the avirulence protein per se, when it is present in synthesised form in a cell of a plant, in no way functions there as an inducer of a plant resistance protein-conveyed pathogen defence reaction. If, however, all different non-functional parts of the same avirulence protein, coded by the nucleic acids integrated in a stable manner in the genome, are present jointly in synthesised manner in a plant cell, all synthesised partial proteins jointly convey the effect of the complete avirulence protein (complementation of the avirulence protein effect) by reacting directly or indirectly with the corresponding resistance protein. The extent of the cell death trigger achieved here, however, is not necessarily comparable with that which could be caused by the reaction of the entire avirulence protein with the corresponding resistance protein. By way of example, the degree of the cell death trigger can thus be increased by the substitution of an individual amino acid in a partial protein, already after complementation, whereas conversely an increased N-terminal deletion of amino acids in a partial protein can lead to a considerable weakening of the cell death trigger, for example. This shows that, with the aid of modifications of the amino acid sequence of a partial protein, the extent of the cell death trigger induced by the complementation, that is to say the efficacy of the inducer, can be controlled already. The intensity of the pathogen defence reaction caused by the transgenic inducer can thus be controlled and predetermined.

Nucleic acids that can be used in accordance with the invention can be extracted from such an avirulence gene of a pathogen which codes for an avirulence protein that
a) finds a corresponding resistance protein in the plant provided for genomic integration or in at least one cell of this plant, with which resistance protein the avirulence protein can react directly or indirectly, and consequently a plant pathogen defence reaction is then induced, and
b) can be broken down into at least two different protein parts, wherein the different parts of the avirulence protein, considered individually, do not constitute inducers of a plant pathogen defence reaction, but react directly or indirectly with the present, corresponding resistance protein when the different parts of the avirulence protein are present together in synthesised form in a cell of the plant provided for genomic integration, and consequently a plant pathogen defence reaction is then induced.

By way of example, an avirulence gene with a nucleotide sequence according to SEQ ID NO: 1, SEQ ID NO: 3 and variants thereof meet the above requirements. Such nucleotide sequences code, for example, for amino acid sequences according to SEQ ID NO: 2 or SEQ ID NO: 4.

Avirulence genes that code for a strong inducer in a plant cell and can therefore efficiently induce HR-conveyed cell death are preferably used.

In addition, a suitable avirulence gene can be modified in accordance with the degradation of the genetic code whilst maintaining the original amino acid sequence of the avirulence protein from the pathogen. Furthermore, before the nucleic acids usable in accordance with the invention are extracted, the nucleotide sequence of a suitable avirulence gene can also be modified, for example in order to change the efficacy, the specificity and/or the activity of the avirulence protein or for example in order to remove an intron that is present. Modifications can be made by addition, substitution or deletion of one or more nucleotides. The implementation of such modifications is well known to a person skilled in the art. In any case, a modified avirulence gene should also code such an avirulence protein that meets the above requirements a) and b). In addition, the nucleotide sequence of a modified avirulence gene hybridises under usual conditions (Sambrook et al. 1989), preferably under stringent conditions, with the non-modified nucleotide sequence or indicates at DNA level a homology of at least 60%, 70%, 80%, 90%, 95%, 96%, 97%, 98% or 99% to the non-modified nucleotide sequence. The coded amino acid sequence has an identity of at least 60%, 70%, 80%, 90%, 95%, 96%, 97%, 98% or 99% or a similarity of at least 60%, 70%, 80%, 90%, 95%, 96%, 97%, 98% or 99% with the non-modified amino acid sequence.

The aforementioned genetic modifications can also be used in order to modify an unsuitable avirulence gene of a pathogen in such a way that it then meets the above requirements a) and b) so as to then obtain therefrom nucleic acids that can be used in accordance with the invention for integration into the genome of a plant and for production of a pathogen-resistant plant according to the invention.

Nucleic acids from a suitable avirulence gene that can be used in accordance with the invention can be reliably identified using a method that comprises the following five steps.
(1) Producing different nucleic acids that comprise fragments of the coding region of an avirulence gene.

This first method step can be performed by means of standard DNA cloning techniques (Sambrook et al. 1989). By way of example, by introducing new translation start and/or stop codons from the 5′ and/or 3′ end, a shortening of the avirulence gene can be achieved. A number of N- and/or C-terminally shortened parts of the avirulence protein can thus then be synthesised in the next method steps (2) and (3).
(2) Transient expression of individual nucleic acids from step (1) under the control of a constitutive promoter in plant cells that provide a resistance protein corresponding to the avirulence protein.

A person skilled in the art can use the conventional and known methods from the prior art for this purpose. By way of example, Schmidt et al., 2004 describes a transient expression system in cells of a plant leaf tissue on the basis of biolistic transfer techniques. The transient expression is to be performed in cells of such a plant type for which a pathogen resistance is to be established. Accordingly, the constitutive promoter is also to be selected in such a way that it is functional in a cell of this plant (for example double 35S promoter). The transient expression is preferably to be limited to cells of such organs, tissues or plant parts that are known as pathogen-typical infection sites.
(3) selection of individual nucleic acids, of which the expression in step (2) has not led to an HR-conveyed cell death.

In order to detect and to quantify the cell death trigger in step (3), a method for detecting the vitality of plant cells can be used. For this purpose, it is possible for example to perform the expression of the nucleic acids produced in step (1) and/or of the complete avirulence gene as reference in the presence of at least two reporter genes, such as the luciferase reporter gene from Photinus pyralis and Renilla reniformis.
(4) Transient co-expression of at least two selected nucleic acids from (3), in each case under the control of a constitutive promoter in plant cells, which provide a resistance protein corresponding to the avirulence protein.

As in step (2), a person skilled in the art can revert to conventional and known methods from the prior art. The necessary demands on the selection of the transformed cells and the usable constitutive promoters correspond to those from step (2).
(5) Identification of at least two selected nucleic acids of which the co-expression in step (4) has led to an HR-conveyed cell death.

In a preferred embodiment, the cell death trigger is comparable with that caused by the expression of the complete avirulence gene under the control of a constitutive promoter. The detection and quantification can be based on the detection method described in step (3).

Nucleic acids identified with the aid of the above method, isolated from an avirulence gene, are suitable for the production of a pathogen-resistant plant according to the invention, since the synthesis products of these nucleic acids are used commonly in a cell of the plant as inducers of a pathogen defence reaction. In order to ensure that this induction takes place as intended or desired, the regulation and control of the expression of the used nucleic acids in the pathogen-resistant plant is to be configured as follows.

The nucleic acids, integrated in a stable manner, of a pathogen-resistant plant according to the invention are each operatively linked to a promoter that regulates the expression of the corresponding nucleic acid. At least one of these promoters is pathogen-inducible, more specifically this promoter is activated as a result of an infection of the plant by the pathogen or by the pathogens with respect to which a resistance in the plant according to the invention is to be established ultimately. This means that the expression of the nucleic acid operatively linked to the pathogen-inducible promoter takes place only as a result of an infection of the plant by the pathogen or the pathogens, and therefore the coded part of the avirulence protein is only then present in cells of the plant in synthesised form. The promoters that are operatively linked to the remaining nucleic acids are characterised in that they have such a specificity that ensures that, at the moment when the part of the avirulence protein is present in synthesised form in an infected cell of the plant under the control of the aforementioned pathogen-inducible promoter, the remaining parts of the avirulence protein are also present in this cell in synthesised form. This is achieved in that the promoters that are operatively linked to the remaining nucleic acids have such a specificity that, with the specified pathogen-inducible promoter, a spatially, temporally and/or otherwise overlapping expression regulation of the operatively linked nucleic acids is ensured. By way of example, three promoters may have an overlapping expression regulation, of which one is fruit tissue-specific, a further one is fruit maturity-specific, and a third is fungal pathogen-specific. An overlapping expression of the nucleic acids operatively linked to the three promoters then takes place only after fungal attack of the fruit and only in the maturing fruit itself. In principle, all known promoters can be used as promoters for regulation of the remaining nucleic acids, for example including constitutive, tissue-specific, organ-specific, storage-induced, development-specific or also pathogen-inducible promoters.

To achieve broad pathogen resistance, the specified pathogen-inducible promoter is to be selected such that it can be induced by as many pathogens or pathogen classes as possible, such as viruses, bacteria, fungi and/or animals. The more specific is one of the used pathogen-inducible promoters, for example for a specific pathogen or a specific part of a pathogen class, the stronger is also the delimitation of the spectrum of pathogens with respect to which an increased resistance is ultimately achieved in the plant according to the invention. Different pathogen-inducible promoters are preferably to be used, since the specificity with respect to abiotic stimuli can thus be increased considerably.

The pathogen-inducible promoter additionally preferably allows an expression of the regulated nucleic acid in a manner delimited locally to the location of a pathogen infection or a use (Strittmatter et al., 1996; Rushton et al, 2002). The use of a pathogen-inducible promoter that is activated directly or indirectly by a pathogenic effector molecule released by numerous pathogens or pathogen classes would also be advantageous. Such an effector molecule, for example, is the known PEP25. In particular, pathogen-inducible promoters that are induced either directly or indirectly as a result of a use can also be used, in particular for the defence of pathogens penetrating into the cell/plant. The use of pathogen-inducible promoters of which the activation is conveyed directly or indirectly through the plant PAMP/MAMP identification is particularly advantageous. Thus, as soon as the pathogen is identified by a pathogen-responsive trans-membrane receptor, that is to say also before or whilst the pathogen infiltrates the cell/plant, the different parts of the avirulence protein in the cell interior are already provided together, whereupon an ETI is triggered as a result of the reaction with the corresponding resistance protein. Due to this “short circuit” between the PAMP/MAMP identification and the ETI, the reaction time of pathogen identification to ETI is considerably reduced and the resistance power is considerably increased.

In a preferred embodiment, at least one promoter for regulation of the expression of the nucleic acids is a synthetic or chimeric promoter. In a particularly preferred embodiment, at least one of the pathogen-inducible promoters is a synthetic or chimeric promoter. The reason for this lies in the fact that, previously, the plant pathogen-inducible promoters were usually used predominantly by pathogen-responsive genes, of which the specificity could be further improved in part by a shortening (Martini et al., 1993). Since these pathogen-responsive genes (for example PR protein genes), however, are not only activated under biotic stress, but also in response to abiotic stress, hormonal changes and diverse development stimuli, the acquisition of a sufficient or exclusive pathogen specificity is technically difficult to implement (Stahl et al., 2006). Synthetic or chimeric promoters, by contrast, merely contain the sequence motifs (for example cis-regulatory elements) from natural, pathogen-inducible promoters that are relevant for the pathogen induction. Sequence motifs for other stimuli were removed, by contrast. The cis-regulatory elements were cloned upstream of a minimal promoter, whereby a functional promoter was produced that has an increased specificity in comparison to the natural promoters, from which the respective cis-regulatory elements were isolated (Rushton et al., 2002).

Diverse cis-regulatory elements for conveying a pathogen inducibility of a promoter are already known from the prior art (for example see WO/00/29592).

In principle, any pathogen-responsive cis-regulatory element can be used in a synthetic or chimeric pathogen-inducible promoter. Such cis-regulatory elements can be present in multiple copies and/or in combination with one another and/or with other cis-regulatory elements in a synthetic or chimeric promoter.

The usable nucleic acids that are suitable for the production of a pathogen-resistant plant according to the invention form, in a manner operatively connected to the specific promoters coordinated with one another, a composition of nucleic acids that comprises at least two nucleic acids for integration in a genome of a plant, wherein the nucleic acids

(i) code for different parts of an avirulence protein and
(ii) are operatively linked to promoters,
and at least one of the promoters is pathogen-inducible, such that, in a cell of the plant, as a result of an infection of the plant by the pathogen, the different parts of the avirulence protein are present in synthesised form and react directly or indirectly with a corresponding resistance protein.

The pathogen resistance of the plant according to the invention is conveyed by the direct or indirect reaction of the synthesised parts of the avirulence protein with a resistance protein already provided in a cell of the plant and corresponding to the avirulence protein (Flor, 1971; Dangl & Jones, 2001; Jones & Dangl, 2006). The resistance gene, which codes for the resistance protein, is either already contained naturally in the genome of the plant according to the invention or has been inserted via gene engineering or breeding methods (Keller et al., 1999; Belbahri et al., 2001).

A plant according to the invention can be of any species from the dicotyledoneae, monocotyledoneae and gymnosperm plants. By way of example, such pants can be selected from the species of the following groups: Arabidopsis, sunflowers, tobacco, sugar beet, cotton, maize, wheat, barley, rice, sorghum, tomatoes, bananas, melons, potatoes, carrots, soys ssp., sugar cane, wine, rye, yeast, rapeseed, lawn grass and forage grass. A plant according to the invention is preferably a plant of the genus Beta. A seed, a part, an organ, a tissue or a cell of the plant according to the invention are also included by the invention.

In order to avoid an unwanted cell death (for example as a result of a hypersensitive reaction) or another negative influencing of the cells of the plant during the production of a plant according to the invention, which could have effects on the agronomic properties of the plant, it should be ensured that, during the production, the different parts of an avirulence protein that lead following reaction with the corresponding resistance protein to a successful induction of a pathogen defence reaction are at no time present in a cell of the plant in synthesised form. Consequently, the usual techniques of plant transformation cannot be used for the integration of the corresponding nucleic acids in a common plant genome, since the execution of these methods causes a pathogen identification by the plant, which causes the activation of the pathogen-inducible promoter (for example Agrobacterium tumefaciens-conveyed transformation), or these methods are so invasive that the injuries caused lead to the induction of a pathogen-inducible promoter (for example biolistic transformation). However, precisely this activation of the pathogen-inducible promoter would, as a trigger at the incorrect moment in time and at the incorrect location, lead in an unintended manner to the induction of the plant pathogen defence reaction.

A suitable production method that bypasses an activation of pathogen-inducible promoters is, for example, the crossing of two transgenic parent plants, wherein each of these parent plants is characterised in that it has been transformed in a stable manner with at least one, but not with all, nucleic acids from the composition of nucleic acids and themselves do not have the pathogen resistance intended for the descendant. In addition, it should be ensured that a parent plant comprises at least the/those nucleic acid(s) from the composition of nucleic acids that the other parent plant does not comprise and does not contain at least one nucleic acid from the composition of nucleic acids that the other parent plant does comprise. By way of example, the two plants could exhibit the following genetic configuration: The first parent plant is characterised by a nucleic acid that is integrated in a stable manner in the genome and that codes for a first part of the avirulence protein and is operatively linked to a pathogen-inducible promoter. The second parent plant is characterised by a nucleic acid that is integrated in a stable manner in the genome and that codes for a second part of the avirulence protein and is operatively linked to a promoter with a specificity that has an expression regulation overlapping with the pathogen-inducible promoter. The first and second parts of the avirulence protein are different. The nucleic acids of the first and second parent plants integrated in a stable manner in the genome are passed on during the crossing to a descendant of the two plants. This produced plant constitutes a pathogen-resistant plant according to the invention. The resistance gene that codes for the resistance protein corresponding to the avirulence protein is present at least in one plant used for the crossing and is passed on again to the produced plant according to the invention during the crossing. Besides the parent plants for producing a pathogen-resistant plant according to the invention, the invention also concerns seeds, parts, organs, tissues or cells of these plants, and also the use of these for the purpose of production of a plant according to the invention.

For the stable genomic integration of the nucleic acids in the respective genome of the parent plant, a person skilled in the art can revert to the methods used conventionally. Here, it is possible for example for the above-mentioned first parent plant itself to be transformed in an Agrobacterium tumefaciens-conveyed manner, since even when this is used for an induction of the pathogen-inducible promoter, the result of the resultant expression of the operatively linked nucleic acid is a non-functional part of the avirulence protein. This part of the avirulence protein alone is not capable, in response to the used transformation method, of acting as an inducer of a pathogen defence reaction.

For a stable inheritance of the nucleic acids and of the resistance gene from the parent plants to the pathogen-resistant plant according to the invention during the course of the crossing process, the parent plants, for example, can be dihaploid or at least homozygous for the respective nucleic acid(s) and/or for the resistance gene. The production of such plants is well known to a person skilled in the art (Gürel et al., 2000).

The plant according to the invention may be a hybrid plant (hybrid) that, besides the increased resistance to at least one pathogen due to the heterosis effect, may also have other advantageous agronomic properties. Such properties are, for example, improved tolerances with respect to abiotic or biotic stress, increased yield, etc. For the production of hybrid plants, it is advantageous to use inbred plants as parent plants. The production of a plant according to the invention in a hybrid system requires the parent plants of the hybrid descendant to be devoid of the pathogen resistance to at least one pathogen conveyed by the synthesis products of the nucleic acids. Only in the hybrids (F1 generation) is this feature expressed. Populations of descendants of the F1 hybrids (F2, F3, etc. generations) tend to lose the pathogen resistance again due to segregation. From commercial viewpoints, such a hybrid system is highly interesting.

Embodiments of the present invention will be described in an exemplary manner with reference to the accompanying figures and sequences:

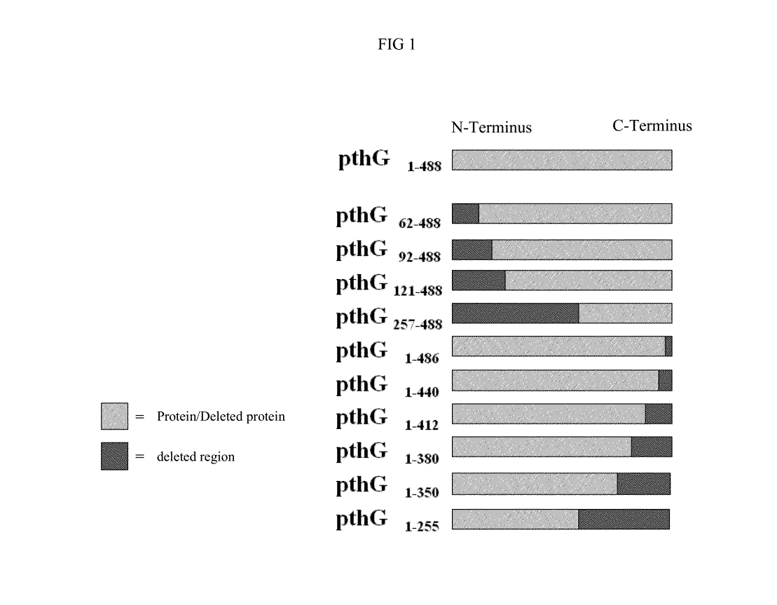

FIG. 1: Illustration of the 5′ and 3′ shortenings in the pthG gene, which have been created for the functional characterisation of the pthG protein. The position of the N- and C-terminal regions deleted in the protein is shown in dark grey, and that of the remaining regions is shown in light grey. The amino acid sequences coded by the DNA fragments are reproduced by subscript numbers (for example pthG₆₂₋₄₈₈ codes for the PthG protein of amino acid position 62 to 488).

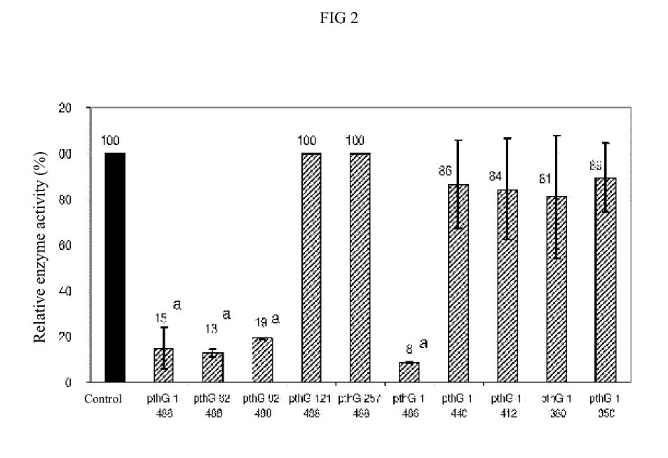

FIG. 2: Detection of non-functional parts of the avirulence protein PthG by transient co-expression of nucleic acids in leaves of a Beta vulgaris plant. The level of the reporter gene activity is a measure for the vitality of the transformed Beta vulgaris cells. Measured values are specified as mean values of 3 tests±SD. The empty vector without pthG gene is used as control. 100% enzyme activity=no cell death, 0% enzyme activity=complete death of the transformed cells. The constructs denoted by a differ from the other constructs in a statistically significant manner.

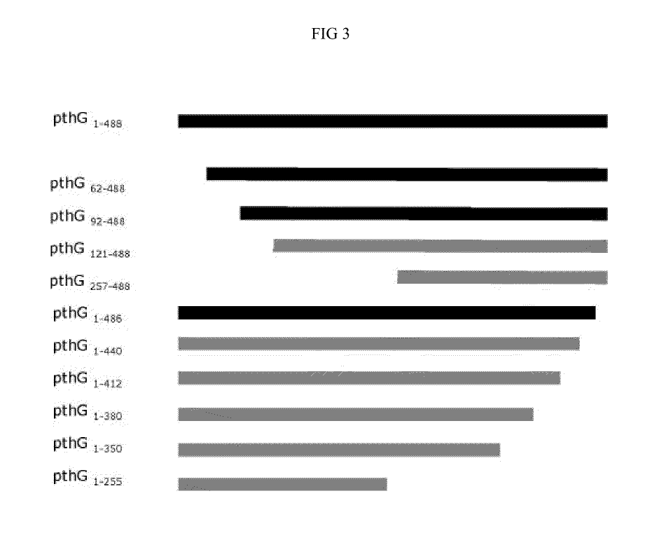

FIG. 3: Schematic illustration of the results for identification of the functional areas of the PthG protein necessary for the cell death trigger. The DNA fragments that can trigger cell death following transient expression are illustrated in black. The shortened DNA fragments of the pthG gene that could no longer trigger cell death are reproduced in light grey.

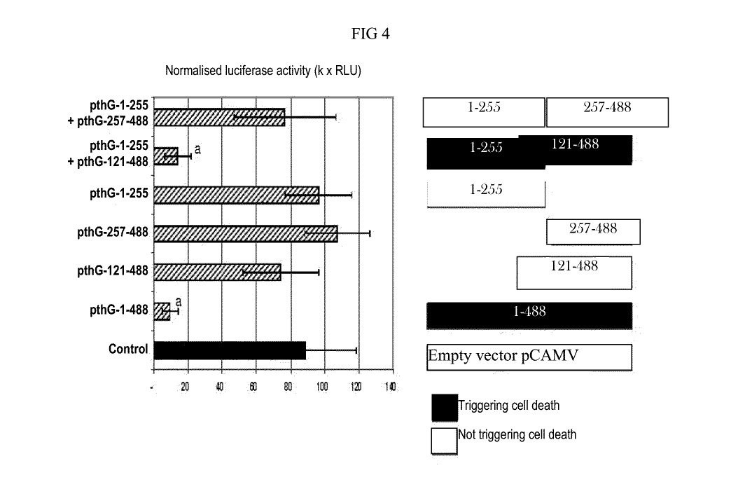

FIG. 4: Detection of the complementation of the avirulence gene function (cell death trigger) by co-expression of shortened inactive pthG gene fragments. The function of the cell death trigger can be restored by co-expression of the DNA sequences pthG₁₋₂₅₅ with the DNA sequence pthG₁₂₁₋₄₈₈. Left illustration: Normalised reporter gene activities (luciferase) following transient expression of pthG gene fragments in sugar beet leaves. The mean value of a representative experiment with 6 biological replicates per construct is illustrated. The empty vector without pthG gene was used as control. 100% enzyme activity=no cell death, 0% enzyme activity=complete death of the transformed cells. The constructs denoted by a differ statistically significantly from the rest of the constructs.

Right illustration: Size and number of the PthG protein fragments expressed during the experiment per batch.

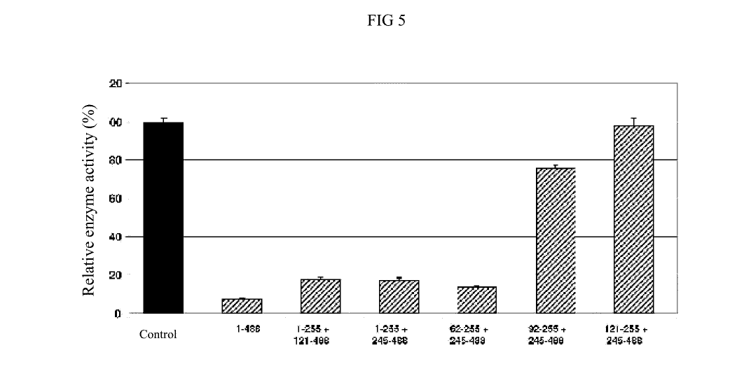

FIG. 5: Detection of the sequences of the pthG gene required as minimum for the complementation by transient co-expression in sugar beet leaves. By combining different pthG gene sequences in the complementation experiment, the intensity of the cell death trigger can be controlled quantitatively. The extent of the relative reporter gene activity is a measure for the vitality of the transformed Beta vulgaris cells. Mean values are specified as mean values of 3 tests±SD. The empty vector without pthG gene was used as control. 100% enzyme activity=no cell death, 0% enzyme activity=complete death of the transformed cells.

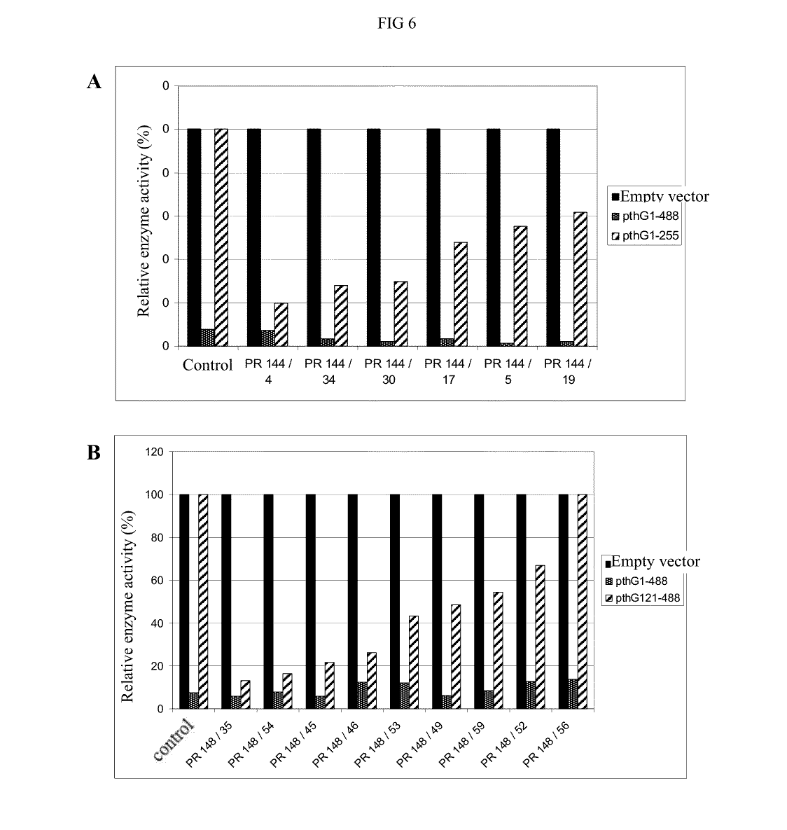

FIG. 6: Functional characterisation of the sugar beets transformed in a stable manner with the sequences pthG₁₂₁₋₄₈₈ and pthG₁₋₂₅₅. The suitability of each independent sugar beet transformant for the cell death trigger and therefore the intensity of the pathogen defence was determined quantitatively by a transient complementation test. The extent of the relative reporter gene activity is a measure for the vitality of the transformed Beta vulgaris cells. The empty vector without pthG gene was used as control. 100% enzyme activity=no cell death, 0% enzyme activity=complete death of the transformed cells.

A. The lines PR144 are transformed with the construct 2×S-2×D-pthG₁₂₁₋₄₈₈-kan.
B. The lines PR148 are transformed with the construct 2×S-2×D-pthG₁₋₂₅₅-kan.

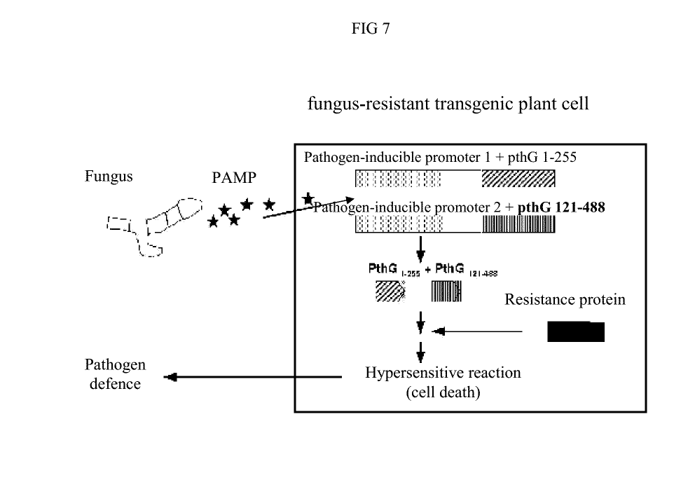

FIG. 7: Schematic illustration of a plant cell in which, by crossing, the two pthG sequences necessary for the complementation have been merged from two independent transgenic parent lines. The expression of the pthG fragments is under the control of two identical or different synthetic pathogen-specific promoters 1 and 2. The co-expression of the PthG protein fragments PthG₁₋₂₅₅ and PthG₁₂₁₋₄₈₈, in reaction with an as yet unknown resistance protein, triggers a hypersensitive reaction (cell death) or a severe defence reaction, which leads to an improved fungal resistance. PAMP=“pathogen-associated molecular pattern” (signal substances that activate pathogen-responsive promoters).

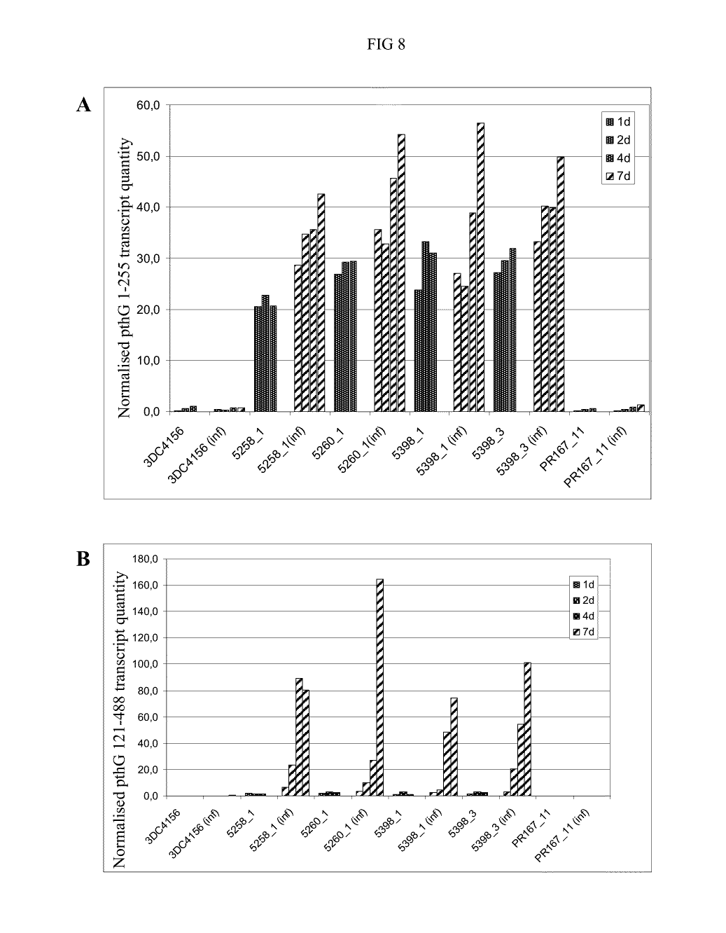

FIG. 8: Detection of the transcription accumulation of the gene pthG₁₋₂₅₅ and pthG₁₂₁₋₄₈₈ in PR144×PR148 crossings by qRT-PCR. Normalised transcription accumulation of pthG₁₋₂₅₅ (A) and pthG₁₂₁₋₄₈₈ (B) in in-vitro plants of the species Beta vulgaris (see also Table 6) and in the control plants, 3DC4156 and PR167/11, on day 0, 1, 2, 4 and 7 following C. beticola infection. Measured values represent mean values of each three biological replicates.

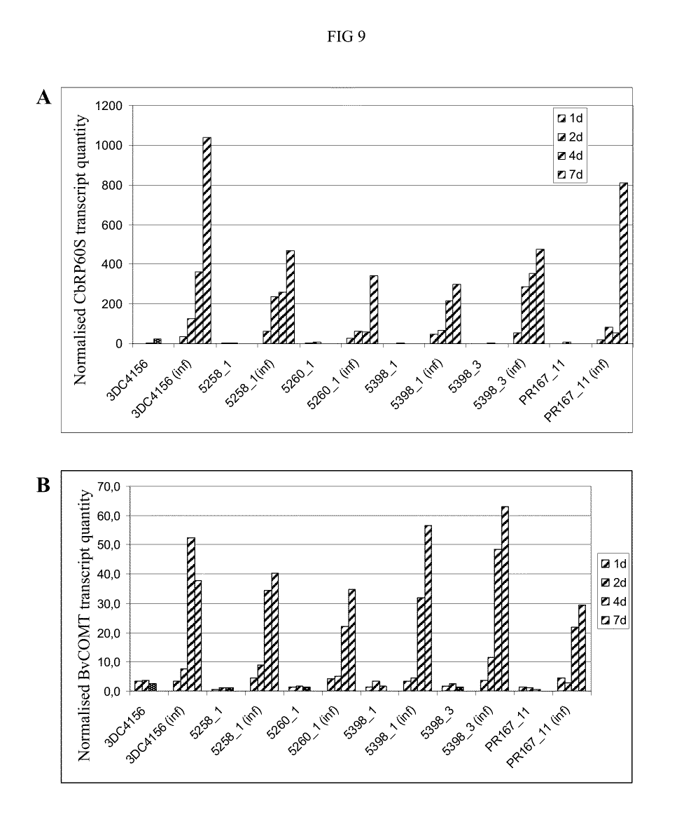

FIG. 9: Detection of the reduction of fungal biomass and of an amplified pathogen defence in PR144×PR148 crossings by qRT-PCR. Normalised transcription accumulation of the C. beticola ribosomal protein gene 60S (A) and of the B. vulgaris gene for the BvCoMT (B) in in-vitro plants from PR144×PR148 crossings (see Table 6) and in the control plants, 3DC4156 and PR167/11, on day 0, 1, 2, 4 and 7 following C. beticola infection. Measured values represent mean values of each three biological replicates.

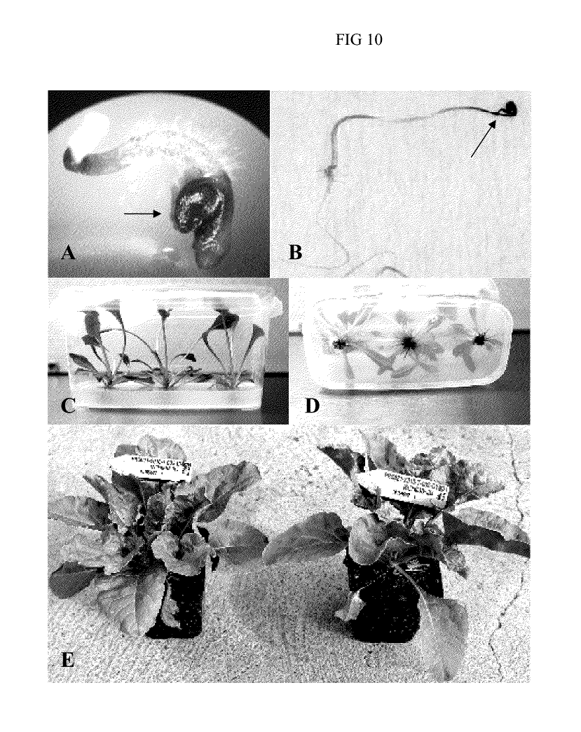

FIG. 10: Different development of PthG crossings following germination and in the greenhouse

A: quick cell death of a sugar beet seedling (within 3 days) from the crossing PR171/19×PR144/4
B: delayed cell death of the seedling (within 8 days) from the crossing PR 171/19×PR144/5;
C: regenerated, vigorous sugar beet plant from the crossing PR171/19×PR144/19.
D: rooted regenerated, vigorous sugar beet plant from the crossing PR171/19×PR144/19.
E: normal growth of descendant from the crossing PR171/19×PR144/19 in the greenhouse. Arrows show seedling tissue in which an undesired cell death trigger took place.

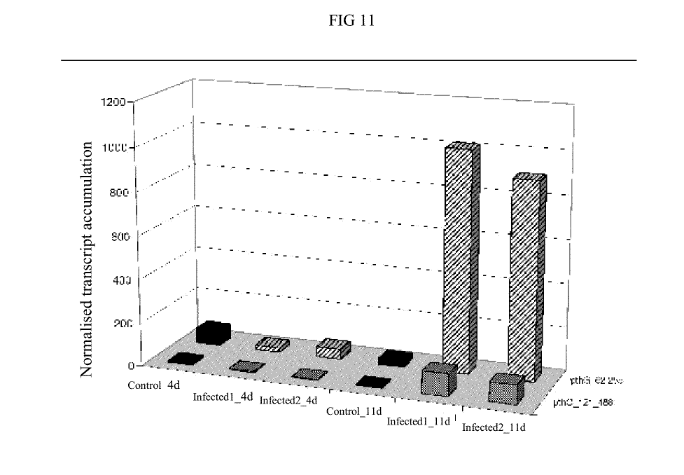

FIG. 11: Detection of the transcription accumulation of the gene pthG₆₂₋₂₅₅ and pthG₁₂₁₋₄₈₈ in the PR171/19×PR144/19 crossing PR5021-2010-T-003 by qRT-PCR.

In the greenhouse, transgenic sugar beets that, following the crossing, carry the promoter gene combinations 4×D-pthG 62-255 and 2×S-2×D-pthG 121-488 exhibit a strong accumulation both of the pthG₆₂₋₂₅₅ and of the pthG₁₂₁₋₄₈₈ transcripts 11 days after the inoculation. The transcription quantities have been normalised in accordance with Weltmeier et al. (2011) against a constitutively expressed sugar beet gene. The analysed plants were clonally produced starting from the lineage PR5021-2010-T-003 and are genetically identical. Control=uninfected PR5021-2010-T-003 plant, Infected 1 and Infected 2=two infected PR5021-2010-T-003 plants.

### SEQUENCES

SEQ ID NO: 1 Nucleotide sequence of the coding area of the bacterial pthG gene from the plasmid pQE60-pthG, which contains a 2.8 kb large genomic BamHI HindIII fragment from Erwinia herbicola pv. gypsophilae.
   SEQ ID NO: 2 Amino acid sequence of the PthG protein
   SEQ ID NO: 3 Nucleotide sequence of the pthG₁₋₄₈₈ gene with flanking NcoI and BamHI interface
SEQ ID NO: 4 Amino acid sequence of the PthG₁₋₄₈₈ protein
SEQ ID NO: 5 Nucleotide sequence (pthG₁₋₂₅₅), coding for partial protein PthG₁₋₂₅₅
SEQ ID NO: 6 Amino acid sequence of the partial protein PthG₁₋₂₅₅
SEQ ID NO: 7 Nucleotide sequence (pthG₆₂₋₂₅₅), coding for partial protein PthG₆₂₋₂₅₅
SEQ ID NO: 8 Amino acid sequence of the partial protein PthG₆₂₋₂₅₅
SEQ ID NO: 9 Nucleotide sequence (pthG₉₂₋₂₅₅), coding for partial protein PthG₉₂₋₂₅₅
SEQ ID NO: 10 Amino acid sequence of the partial protein PthG₉₂₋₂₅₅
SEQ ID NO: 11 Nucleotide sequence (pthG₁₂₁₋₂₅₅), coding for partial protein PthG₁₂₁₋₂₅₅
SEQ ID NO: 12 Amino acid sequence of the partial protein PthG₁₂₁₋₂₅₅
SEQ ID NO: 13 Nucleotide sequence (pthG₁₂₁₋₄₈₈), coding for partial protein PthG₁₂₁₋₄₈₈
SEQ ID NO: 14 Amino acid sequence of the partial protein PthG₁₂₁₋₄₈₈
SEQ ID NO: 15 Nucleotide sequence (pthG₉₂₋₄₈₈), coding for partial protein PthG₉₂₋₄₈₈
SEQ ID NO: 16 Amino acid sequence of the partial protein PthG₉₂₋₄₈₈
SEQ ID NO: 17 Nucleotide sequence (pthG₁₆₂₋₄₈₈), coding for partial protein PthG₁₆₂₋₄₈₈
SEQ ID NO: 18 Amino acid sequence of the partial protein PthG₁₆₂₋₄₈₈
SEQ ID NO: 19 Nucleotide sequence (pthG₂₀₅₋₄₈₈), coding for partial protein PthG₂₀₅₋₄₈₈
SEQ ID NO: 20 Amino acid sequence of the partial protein PthG₂₀₅₋₄₈₈
SEQ ID NO: 21 Nucleotide sequence (pthG₂₄₅₋₄₈₈), coding for partial protein PthG₂₄₅₋₄₈₈
SEQ ID NO: 22 Amino acid sequence of the partial protein PthG₂₄₅₋₄₈₈
SEQ ID NO: 23 Nucleotide sequence (pthG₂₅₃₋₄₈₈), coding for partial protein PthG₂₅₃₋₄₈₈
SEQ ID NO: 24 Amino acid sequence of the partial protein PthG₂₅₃₋₄₈₈
SEQ ID NO: 25 Nucleotide sequence (pthG_{G+256-488}), coding for partial protein PthG_{G+256-488}
SEQ ID NO: 26 Amino acid sequence of the partial protein PthG_{G+256-488}
SEQ ID NO: 27 Nucleotide sequence (pthG₂₅₇₋₄₈₈), coding for partial protein PthG₂₅₇₋₄₈₈
SEQ ID NO: 28 Amino acid sequence of the partial protein PthG₂₅₇₋₄₈₈
SEQ ID NO: 29 Nucleotide sequence (pthG₁₋₃₅₀), coding for partial protein PthG₁₋₃₅₀
SEQ ID NO: 30 Amino acid sequence of the partial protein PthG₁₋₃₅₀
SEQ ID NO: 31 Nucleotide sequence (pthG₁₋₃₈₀), coding for partial protein PthG₁₋₃₈₀
SEQ ID NO: 32 Amino acid sequence of the partial protein PthG₁₋₃₈₀
SEQ ID NO: 33 Nucleotide sequence (pthG₁₋₄₁₂), coding for partial protein PthG₁₋₄₁₂
SEQ ID NO: 34 Amino acid sequence of the partial protein PthG₁₋₄₁₂
SEQ ID NO: 35 Nucleotide sequence (pthG₁₋₄₄₀), coding for partial protein PthG₁₋₄₄₀
SEQ ID NO: 36 Amino acid sequence of the partial protein PthG₁₋₄₄₀
SEQ ID NO: 37 Nucleotide sequence (pthG₁₋₄₈₆), coding for partial protein PthG₁₋₄₈₆
SEQ ID NO: 38 Amino acid sequence of the partial protein PthG₁₋₄₈₆
SEQ ID NO: 39 Nucleotide sequence S549, for a primer
SEQ ID NO: 40 Nucleotide sequence S544, for a primer
SEQ ID NO: 41 Nucleotide sequence S558, for a primer
SEQ ID NO: 42 Nucleotide sequence S550, for a primer
SEQ ID NO: 43 Nucleotide sequence S551, for a primer
SEQ ID NO: 44 Nucleotide sequence S545, for a primer
SEQ ID NO: 45 Nucleotide sequence S552, for a primer
SEQ ID NO: 46 Nucleotide sequence S561, for a primer
SEQ ID NO: 47 Nucleotide sequence S560, for a primer
SEQ ID NO: 48 Nucleotide sequence S559, for a primer
SEQ ID NO: 49 Nucleotide sequence S553, for a primer
SEQ ID NO: 50 Nucleotide sequence S562, for a primer
SEQ ID NO: 51 Nucleotide sequence S554, for a primer
SEQ ID NO: 52 Nucleotide sequence S1420, for a primer for qRT-PCR determination of the ribosomal protein 60S from C. beticola
SEQ ID NO: 53 Nucleotide sequence S1421, for a primer for qRT-PCR determination of the ribosomal protein 60S from C. beticola
SEQ ID NO: 54 Nucleotide sequence (pthG₉₂₋₁₂₀), coding for partial protein PthG₉₂₋₁₂₀
SEQ ID NO: 55 Amino acid sequence of the partial protein PthG₉₂₋₁₂₀
SEQ ID NO: 56 Nucleotide sequence (pthG₄₄₁₋₄₈₆), coding for partial protein PthG₄₄₁₋₄₈₆
SEQ ID NO: 57 Amino acid sequence of the partial protein PthG₄₄₁₋₄₈₆
   Agrobacterium tumefaciens-Conveyed Transformation of a Sugar Beet Plant (Beta vulgaris) with the Complete Avirulence Gene pthG from Erwinia herbicola pv. Gypsophilae

The avirulence gene pthG (pathogenicity gene on gypsophila, SEQ ID NO: 1) codes for the avirulence protein PthG 488 amino acids in size (SEQ ID NO: 2) and was isolated from the pathogen Erwinia herbicola pv. gypsophilae (Pantoea agglomerans pv. gypsophilae) (Ezra et al., 2000). The pthG gene acts as a virulence factor in baby's breath (gypsophilia). In addition, it codes for a highly effective avirulence protein, which triggers a hypersensitive reaction in all examined Beta species (Beta vulgaris, Beta patula, Beta webbiana, Beta macrocarpa, Beta patellaris, Beta corolliflora, Beta lomatogona) and thus prevents an infection of Beta species by Erwinia herbicola pv. gypsophilae (Ezra et al., 2004). The pthG gene is thus an avirulence gene with a broad host range that reacts with an unknown resistance gene conserved in Beta.

The gene pthG₁₋₄₈₈ (SEQ ID NO: 3) was linked operatively to the currently most suitable synthetic pathogen-inducible promoter, comprising the combination of cis-regulatory elements 2×S-2×D (see WO/00/29592), referred to hereinafter as synthetic promoter 2×S-2×D. The transformation of the construct 2×S-2×D-pthG₁₋₄₈₈-kan in sugar beet cells, performed in accordance with Lindsey & Gallois, 1990, led to a temporary activation of the synthetic promoter due to the used Agrobacterium tumefaciens bacteria and therefore to the death of the transformed plant cells following the expression of the avirulence gene. The regeneration of a living sugar beet plant was not possible with use of 3 different sugar beet genotypes in repeated tests. The transformation of the synthetic promoter 2×S-2×D in combination with the luciferase gene led, by contrast, to 1-15 transformants per test. This result showed that the luciferase gene can indeed be transformed into sugar beets, however the entire gene pthG₁₋₄₈₈ (SEQ ID NO: 3) cannot (Table 1).

TABLE 1
Comparison of the transformability of the gene pthG₁₋₄₈₈ with the
Luc gene in sugar beets. The pthG gene and Luc gene are both under
the control of the synthetic promoter 2xS-2xD in the otherwise identical
binary vectors 2xS-2xD-pthG-kan and 2xS-2xD-luc-kan. The number of
independent transgenic plants obtained per test is presented, and
the used sugar beet genotype is shown in brackets.

| | Construct | Construct |
|---|---|---|
| | 2xS-2xD-pthG₁₋₄₈₈-kan | 2xS-2xD-luc-kan |
| Test 1 | 0 | 17 |
| | (3DC4156 and 3TC4174) | (3DC4156) |
| Test 2 | 0 | 1 |
| | (3D0018) | (3DC4156) |
| Test 3 | 0 | 5 |
| | (3DC4156 and 3TC4174) | (3TC4174) |

### Identification of the Functional Areas of the PthG Protein Necessary for Cell Death Trigger

The coding area of the pthG₁₋₄₈₈ was shortened with insertion of new translation start and stop codons from the 5′ and 3′ end, such that ten N- and C-terminally shortened pthG proteins could be synthesised (FIG. 1). In the case of the N-terminal deletions, the first 61, 91, 120 and 256 amino acids were removed. The gene fragments produced were denoted as pthG₆₂₋₄₈₈, pthG₉₂₋₄₈₈, and pthG121₄₈₈, pthG₂₅₇₋₄₈₈. To this end, the specified fragments were amplified by PCR with use of the primer pairs S549/S544 (SEQ ID NO: 39/SEQ ID NO: 40), S558/S544 (SEQ ID NO: 41/SEQ ID NO: 40), S550/S544 (SEQ ID NO: 42/SEQ ID NO: 40) and S551/S544 (SEQ ID NO: 43/SEQ ID NO: 40) and of the starting plasmid pQE60-pthG with the aid of the Pfu polymerase. The PCR conditions were as follows:

Pfu-PCR (50 μl batch):

| | |
|---|---|
| 10 x Pfu Ultra High Fidelity Buffer | 5 μl |
| dNTPs (each 10 mM) | 5 μl |
| Pfu Ultra High Fidelity Polymerase (1 U/μl) | 1 μl |
| sense primer (20 μM) | 0.5 or 1 μl |
| antisense primer (20 μM) | 0.5 or 1 μl |
| DNA (1-100 ng/μl) | 4 μl |
| bidistilled H₂O | 33 or 34 μl |

Where necessary, MgCl₂ was added to some PCR amplifications. Here, concentrations from 1 V to 4 V were added per PCR.

PCR program Pfu gen. DNA:

| 1^{st} cycle | 2 min 95° | C. (initial denaturation) |
|---|---|---|
| 2^{nd} (25^{th}-35^{th}) cycle | 30 sec 95° | C. (denaturation) |
| | 30 sec 58° C.-60° C. (annealing) | |
| | 2 min 72° | C. (DNA synthesis) |
| Terminal Extension | 10 min 72° | C. |
| End temperature | 10° | C. |

The 5′ primers S549 (SEQ ID NO: 39), S558 (SEQ ID NO: 41), S550 (SEQ ID NO: 42) and S551 (SEQ ID NO: 43) contain an NcoI (CCATGG) interface, with which the N-terminal deletions were provided with a start methionine. The primer S544 (SEQ ID NO: 40) has a BamHI interface after the stop codon of the gene pthG₁₋₄₈₈. By cutting with the restriction enzymes NcoI and BamHI, the DNA fragments S549/S544 (SEQ ID NO: 39/SEQ ID NO: 40), S558/S544 (SEQ ID NO: 41/SEQ ID NO: 40), S550/S544 (SEQ ID NO: 42/SEQ ID NO: 40) and S551/S544 (SEQ ID NO: 43/SEQ ID NO: 40) could be cloned into the vector p70S-165-#176-NcoI (vector known from WO/2006/128444) and placed under the expression control of the double 35S promoter.

In the case of the C-terminal deletions, the last 2, 48, 76, 108, 138 and 233 amino acids were removed. The gene fragments created were named pthG₁₋₄₈₆, pthG₁₋₄₄₀, pthG₁₋₄₁₂, pthG₁₋₃₈₀, pthG₁₋₃₅₀ and pthG₁₋₂₅₅. To this end, the aforementioned DNA fragments were amplified and cloned by PCR with use of the primer pairs S545/S552 (SEQ ID NO: 44/SEQ ID NO: 45), S545/S561 (SEQ ID NO: 44/SEQ ID NO: 46), S545/S560 (SEQ ID NO: 44/SEQ ID NO: 47), S545/S559 (SEQ ID NO: 44/SEQ ID NO: 48), S545/S553 (SEQ ID NO: 44/SEQ ID NO: 49), S545/S554 (SEQ ID NO: 44/SEQ ID NO: 51) and S545/S562 (SEQ ID NO: 44/SEQ ID NO: 50) as described for the N-terminal deletions.

The reduced pthG variants and the complete gene pthG₁₋₄₈₈ were expressed under the control of the double 35S promoter by transient ballistic transformation with use of a PDS-1000 gene gun (BioRad, Munich, Germany) in the presence of two luciferase reporter genes from Photinus pyralis and Renilla reniformis. With the aid of the reporter genes, it was possible to measure the vitality of the transformed cells. The transient ballistic transformation was performed as follows:

### Preparation of Macrocarrier Dip Solution

60 mg of gold (gold powder, Au type 200-03 (Heraeus GmbH, Hanau, Germany) were weighed into an Eppendorf reaction vessel, then 1 ml 70% EtOH was added and the mixture was mixed for 5 min and then left to stand for 15 min. The gold was sedimented by short centrifugation (approximately 5 sec) (Pico Fuge®, Stratagene, Amsterdam) and the supernatant was rejected. The gold was resuspended in 1 ml of bidist. H₂O and mixed for 1 min, left to stand for 1 min and sedimented again (5 sec), the supernatant was rejected and the sediment was resuspended in 1 ml of bidist. H₂O. This washing step was repeated a total of three times. The washed gold was ultimately taken up in 1 ml 50% glycerol.

Charging the Macrocarrier with DNA (for 6 Shot)

For the transformation, only plasmid DNA was used, which had been purified by silica membrane column and set to a concentration of 1 μg/μl. The plasmid p70S luc with the luciferase gene from Photinus pyralis was used as reporter gene construct, and p70S ruc with the luciferase gene of Renilla reniformis was used as normalisation vector.

### Batch for Effector Gold

The gold was mixed for at least 5 min and 2.5 μl of effector plasmid DNA (1 μg/μl) and 2.5 μl p70S luc plasmid DNA were transferred into an Eppendorf reaction vessel and mixed, 25 μl of gold suspension were added; here, it was important to mix the suspension constantly. In addition, 25 μl CaCl₂ (2.5 M) and 10 μl spermidine (0.1 M) were added. The batch was mixed for 3 min, then left to rest for 1 min, the gold was sedimented by short centrifugation (2-3 sec), and the supernatant was removed and discarded. The sediment was washed with 70 μl 70% EtOH and then with 70 μl 100% EtOH, then taken up in 24 μl 100% EtOH and mixed well.

### Batch for Normalisation Gold

The procedure was identical to that for effector gold, however the quantities introduced differ. 5 μl p70S ruc DNA (1 μg/μl) were transferred into an Eppendorf reaction vessel and 50 μl gold suspension, 50 μl CaCl₂ (2.5 M) and 20 μl spermidine (0.1 M) were added. The sediment was washed with 140 μl 70% EtOH and then with 140 μl 100% EtOH, and was then taken up in 48 μl 100% EtOH and mixed well.

In order to be able to use the normalisation gold and compare the different batches with one another, the quantities of normalisation gold were increased in accordance with the number of repetitions.

40 μl of normalisation gold and 10 μl of effector gold were added, then 6 μl of each suspension were distributed uniformly in the middle of the macrocarrier using a 10 μl Gilson pipette. The macrocarriers were positioned beforehand in macrocarrier holders. Once the gold was dried by evaporating off the EtOH, the macrocarriers were able to be used for the bombardment.

The bombardment of the sugar beet leaves with the DNA-charged gold particles and the measurement of the dual luciferase activities were performed as described (Schmidt et al., 2004). The expression of the functional pthG fragments by the double 35S promoter triggers a hypersensitive reaction in the sugar beet cells transiently transformed with the effector gold, thus leading to local cell death. The local cell death of the transformed cells also prevents the expression of the luciferase gene from Photinus pyralis (p70S luc) co-transformed with the effector gold. The measurement of the luciferase of Renilla reniformis shot with the aid of the normalisation gold into the leaf tissue allows a normalisation of the bombardment tests. Compared with the control batch, which contains only the empty vector pCaM-V2 (vector known from WO/2006/128444) instead of a pthG construct, the cell death-triggering effect of the pthG fragments can thus be measured.

The deletion experiments and subsequent function tests demonstrated that, besides the starting protein PthG₁₋₄₈₈, the N-terminal deletions PthG₆₂₋₄₈₈ and PthG₉₂₋₄₈₈ also have a strong cell death-triggering effect. By contrast, the protein part pthG₁₂₁₋₄₈₈, similarly to the protein part PthG₂₅₇₋₄₈₈, no longer triggers cell death, and therefore the protein region of amino acid position 92-120 (SEQ ID NO: 55) is necessary for cell death trigger, whereas the protein region of position 1-91 is unnecessary for cell death trigger. In the case of the C-terminal deletions, the protein part PthG₁₋₄₈₆ also triggered a strong cell death. By contrast, the protein parts PthG₁₋₄₄₀, PthG₁₋₄₁₂, PthG₁₋₃₈₀ and PthG₁₋₃₅₀ no longer triggered any cell death. The amino acid sequence of position 441-486 is thus necessary for the cell death trigger, but the sequence of position 487-488 is not (FIG. 2 and FIG. 3).

Complementation of the Avirulence Gene Function by Co-Expression of Reduced Inactive pthG Gene Fragments

Since the deletion analyses have shown that two sequence portions in the region of amino acid position 92-120 and 441-486 are a precondition for cell death trigger, the inactive partial fragments were transiently co-expressed in sugar beet leaves by the ballistic test. Whereas the nucleic acids, expressed individually, triggered no cell death, the co-expression of the nucleic acids of pthG₁₋₂₅₅ and pthG₁₂₁₋₄₈₈ led to a strong cell death, which is comparable to the cell death that is triggered by the complete protein PthG₁₋₄₈₈. Due to the joint expression of the protein parts PthG₁₋₂₅₅ and PthG₁₂₁₋₄₈₈, there was a complementation of the avirulence protein effect and an induction of the hypersensitive reaction (FIG. 4). By contrast, the co-expression of the nucleic acids of pthG₁₋₂₅₅ and pthG₂₅₇₋₄₈₈ led to no cell death and therefore to no reproduction of the avirulence gene function. The difference between the co-expression of pthG₁₋₂₅₅ and pthG₁₂₁₋₄₈₈ compared with pthG₁₋₂₅₅ and pthG₂₅₇₋₄₈₈ lies in the fact that, in the case of successful complementation, the amino acid sequences of the two protein fragments overlap in part.

The results are surprising in many ways. On the one hand, with other avirulence gene proteins the domain triggering cell death is always only assigned one protein portion, as is known, and on the other hand an intramolecular complementation of the avirulence function is not known.

Identification of the Sequences of the pthG Gene Required as Minimum for the Complementation

The prior work regarding the expression of the two-part Avr gene has shown that the molecules PthG₁₋₂₅₅ and PthG₁₂₂₋₄₈₈ trigger cell death in cells of sugar beet leaves following co-expression. As a result of continuing works, it was possible to identify the sequences required as minimum which are necessary for the functional complementation.

To this end, the C-terminally deleted protein PthG₁₋₂₅₅ was first shortened further starting from the N-terminus. The newly created nucleic acids pthG₆₂₋₂₅₅, pthG₉₂₋₂₅₅, pthG₁₂₁₋₂₅₅ and pthG₁₋₂₅₅ were co-transformed in sugar beet leaves together with the nucleic acid pthG₁₂₁₋₄₈₈ in three tests. The reduction of the molecules led in combination with pthG₁₂₁₋₄₈₈ to a staggered decrease of the cell death trigger. Combined, the protein parts coded by pthG₁₋₂₅₅ and pthG₁₂₁₋₄₈₈ triggered the strongest cell death, which was almost as strong as the cell death by the complete protein PthG₁₋₄₈₈. The protein parts coded by pthG₆₂₋₂₅₅ and pthG₉₂₋₂₅₅ in combination with PthG₁₂₂₋₄₈₈ triggered a weaker cell death, whereas pthG₁₂₁₋₂₅₅ was inactive during the co-expression (Table 2). On the whole, an activity decrease from pthG₁₋₂₅₅ via pthG₆₂₋₂₅₅ to pthG₉₂₋₂₅₅ was observed.

TABLE 2
Delimitation of the amino acid regions necessary for cell
death trigger in the region 1-255 of the avirulence protein
divided in two (+ indicates the intensity of the
triggered cell death; − indicates no cell death).

| | Cell death |
|---|---|
| PthG₁₋₄₈₈ | + + + + |
| PthG₁₋₂₅₅ + PthG₁₂₂₋₄₈₈ | + + + to + + + + |
| PthG₆₂₋₂₅₅ + PthG₁₂₂₋₄₈₈ | + + |
| PthG₉₂₋₂₅₅ + PthG₁₂₂₋₄₈₈ | + |
| PthG₁₂₁₋₂₅₅ + PthG₁₂₂₋₄₈₈ | − |
| PthG₁₂₂₋₄₈₈ | − |

The reduction of the molecule PthG₁₂₁₋₄₈₈ occurred from the N-terminus. The newly created nucleic acids pthG₁₆₂₋₄₈₈, pthG₂₀₅₋₄₈₈, pthG₂₄₅₋₄₈₈, pthG₂₅₃₋₄₈₈, pthG_{G+256-488} and pthG₁₂₁₋₄₈₈ were co-transformed together with the nucleic acid pthG₁₋₂₅₅ in three tests in cells of sugar beet leaves. In the case of the nucleic acid of pthG_{G+256-488}, the coding region was modified such that the amino acid sequence PthG_{G+256-488} was extended at the N-terminus by a glycine. The protein parts PthG₁₆₂₋₄₈₈, PthG₂₀₅₋₄₈₈ and PthG₂₄₅₋₄₈₈ were just as effective as the starting molecule PthG₁₂₁₋₄₈₈ in terms of the cell death trigger. A further reduction to the sequence PthG₂₅₃₋₄₈₈ led to a loss of function (Table 3).

TABLE 3
Delimitation of the amino acid regions necessary for cell
death trigger in the region 121-488 of the avirulence
protein divided in two (+ indicates the intensity
of the triggered cell death; − indicates no cell death).

| | Cell death |
|---|---|
| PthG₁₋₄₈₈ | + + + + |
| PthG₁₋₂₅₅ + PthG₁₂₁₋₄₈₈ | + + + to + + + + |
| PthG₁₋₂₅₅ + PthG₁₆₂₋₄₈₈ | + + + to + + + + |
| PthG₁₋₂₅₅ + PthG₂₀₅₋₄₈₈ | + + + to + + + + |
| PthG₁₋₂₅₅ + PthG₂₄₅₋₄₈₈ | + + + to + + + + |
| PthG₁₋₂₅₅ + PthG₂₅₃₋₄₈₈ | − |
| PthG₁₋₂₅₅ + PthG_{G+256-488} | − |
| PthG₁₋₂₅₅ + PthG₂₅₇₋₄₈₈ | − |

Whereas the amino acid sequence of position 121-244 is dispensable, the sequence portion of PthG₂₄₅₋₄₈₈ in combination with pthG₁₋₂₅₅ is necessary for the successful complementation. A precondition for successful complementation of the effect triggering cell death is a small overlap of the amino acid sequences by the two PthG partial fragments, which is 11 amino acids in the case of PthG₁₋₂₅₅ and PthG₂₄₅₋₄₈₈. An overlap of 3 amino acids as in the case of PthG₁₋₂₅₅ and PthG₂₅₃₋₄₈₈ is inadequate. In a further series of co-expression experiments, the efficacy of the sequences PthG₆₂₋₂₅₅ and PthG₂₄₅₋₄₈₈ identified as being necessary as a minimum was compared directly with that of PthG₁₋₂₅₅ and PthG₁₂₁₋₄₈₈. The molecules PthG₆₂₋₂₅₅ and PthG₂₄₅₋₄₈₈, with co-expression, triggered a cell death just as strong as that triggered by the jointly expressed proteins PthG₁₋₂₅₅ and PthG₁₂₁₋₄₈₈ (FIG. 5).

A weaker cell death was triggered by use of the molecules PthG₉₂₋₂₅₅ and PthG₂₄₅₋₄₈₈. The intensity of the cell death induction can be modulated by the use of PthG₆₂₋₂₅₅ or of PthG₉₂₋₂₅₅.

### Functional Analysis and Selection of 2×S-2×D-PthG₁₋₂₅₅ and 2×S-2×D-PthG₁₂₁₋₄₈₈ Parent Plants for Crossings

Identified and isolated deletion constructs pthG₁₋₂₅₅ and pthG₁₂₁₋₄₈₈ were operatively linked with different promoters, for example also with synthetic pathogen-inducible promoters 2×S-2×D and 4×D responsive to Cercospora beticola. With the binary vectors 2×S-2×D-pthG₁₋₂₅₅-kan and 2×S-2×D-pthG₁₂₁₋₄₈₈-kan, sugar beet plants were then transformed separately in each case (Lindsey & Gallois, 1990). In the case of used pathogen-inducible promoters as well as constitutive promoters, it was possible to produce stable, viable transformants with the constructs comprising pthG₁₋₂₅₅ or pthG₁₂₁₋₄₈₈ in spite of the activation or activity of these promoters during the A. tumefaciens-conveyed transformation, since the non-functional partial proteins PthG₁₋₂₅₅ and PthG₁₂₁₋₄₈₈ did not function as inducers of cell death in their respective transformants.

Sugar beet transformants, which quickly and sufficiently produced the recombinant partial proteins, for example following an artificial C. beticola infection, could be identified by qRT-PCR. The artificial infection of sugar beets, RNA isolation, cDNA synthesis and qRT-PCR were performed as described (Weltmeier et al., 2011). Ideally, the suitability of a transgenic line for cell death trigger was determined quantitatively by a ballistic transient complementation test. To this end, the number of genomic integrations of the pthG gene fragments is first determined by a Southern Blot analysis, as described in Stahl et al., 2004 for sugar beets.

The transgenic lines that have just T-DNA integration and therefore a pthG gene under the control of a synthetic promoter were selected for the complementation tests. Although the used synthetic promoters are activated very specifically by pathogen attack, these promoters are also wound-inducible (Rushton et al., 2002). This wound inducibility was used selectively in the ballistic transformation. Leaves of transgenic lines grown in a greenhouse were in each case transiently ballistically transformed a) with the empty vector pCaMV-2 as negative control, b) with the complete pthG₁₋₄₈₈ gene under the control of the double 35S promoter (construct 70S-pthG₁₋₄₈₈) as positive control, and c) with the complementing partial fragment pthG₁₂₁₋₄₈₈ or pthG₁₋₂₅₅ under the control of the double 35S promoter. The normalised reporter gene activity obtained was set equal to 100% as reference.

Whereas the construct 70S-pthG₁₋₄₈₈ as positive control triggered a strong cell death, such that only 1-14% normalised enzyme activity could be measured for all tested lines, the transient transformation with the complementary pthG gene fragment led to significantly different enzyme activities in accordance with the analysed transgenic line (FIG. 6A, Table 4). The transgenic PR144 lines created with the construct 2×S-2×D-pthG₁₂₁₋₄₈₈kan demonstrated a normalised enzyme activity of 20, 28, 30, 48, 55, 62 and 100% following the complementation with the construct 70S-pthG₁₋₂₅₅. For the transgenic PR148 lines obtained with the construct 2×S-2×D-pthG₁₋₂₅₅-kan, a normalised enzyme activity of 13, 16, 22, 26, 43, 49, 55, 67 and 100% was ascertained following the complementation with the construct 70S-pthG₁₂₁₋₄₈₈ (FIG. 6B, Table 4). The different extent of the induction of cell death or a hypersensitive reaction in different transgenic lines of identical genetics, which each have only an integration of the same construct, was supposedly attributed to the influence of the integration site. The integration of T-DNA of a construct occurs randomly with Agrobacterium tumefaciens-conveyed transformation, as is known. By functional analysis, an entire spectrum of transgenic plants was now available, said plants being capable of inducing a hypersensitive reaction in a graduated manner and being selectable for the crossings.

TABLE 4
Functional characterisation of transgenic 2xS-2xD-pthG₁₋₂₅₅ and 2xS-2xD-pthG₁₂₁₋₄₈₈
sugar beets by ballistic transient complementation. All transgenic lines have just
pthG integration in the genome. The relative enzyme activity of the luciferase
gene co-transformed with the effector genes pthG₁₋₄₈₈, pthG₁₋₂₅₅ and pthG₁₂₁₋₄₈₈ was
determined (100% = no cell death, 0% = very strong cell death, n.d. = not determined).

| | | Transient complementation | | | |
|---|---|---|---|---|---|
| | Transgenic line | relative normalised enzyme activity (%) | | | |
| Sugar beet # | Construct | Empty vector | pthG₁₋₄₈₈ | pthG₁₋₂₅₅ | pthG₁₂₁₋₄₈₈ |
| Control | | 100 | 8 | 100 | 100 |
| not transgenic | | | | | |
| PR 144/4 | 2xS-2xD-pthG-₁₂₁₋₄₈₈ -kan | 100 | 7 | 20 | n.d. |
| PR 144/34 | | 100 | 4 | 28 | n.d. |
| PR 144/30 | | 100 | 2 | 30 | n.d. |
| PR 144/17 | | 100 | 4 | 48 | n.d. |
| PR 144/5 | | 100 | 1 | 55 | n.d. |
| PR 144/19 | | 100 | 2 | 62 | n.d. |
| PR 148/35 | 2xS-2xD-pthG-₁₋₂₅₅ -kan | 100 | 6 | n.d. | 13 |
| PR 148/54 | | 100 | 8 | n.d. | 16 |
| PR 148/45 | | 100 | 6 | n.d. | 22 |
| PR 148/46 | | 100 | 12 | n.d. | 26 |
| PR 148/53 | | 100 | 12 | n.d. | 43 |
| PR 148/49 | | 100 | 6 | n.d. | 49 |
| PR 148/59 | | 100 | 9 | n.d. | 55 |
| PR 148/52 | | 100 | 13 | n.d. | 67 |
| PR 148/56 | | 100 | 14 | n.d. | 100 |

The transgenic lines PR171 and PR173, which had been transformed with the 4×D promoter in combination with the sequences pthG₆₂₋₂₅₅ and pthG₂₄₅₋₄₈₈ required as minimum, were analysed in the same way in terms of function. Comparably to the results with the PR144 and PR148 plants, the 4×D-pthG₆₂₋₂₅₅ and 4×D-pthG₂₄₅₋₄₈₈ plants demonstrated a large range of cell death induction following the transient complementation with the constructs 70S-pthG₂₄₅₋₄₈₈ and 70S-pthG₆₂₋₂₅₅ (Table 5).

TABLE 5
Functional characterisation of transgenic 4xD-pthG₆₂₋₂₅₅ and 4xD-pthG₂₄₅₋₄₈₈
sugar beets by ballistic transient complementation. All transgenic lines exhibit
only pthG integration in the genome in accordance with Southern Blot analy-
sis. The relative enzyme activity of the luciferase genes co-transformed with
the effector genes pthG₁₋₄₈₈, pthG₂₄₅₋₄₈₈ and pthG₆₂₋₂₅₅ was determined.
100% = no cell death, 0% = very strong cell death. n.d. = not determined

| | | Transient complementation | | | |
|---|---|---|---|---|---|
| | Transgenic line | relative normalised enzyme activity (%) | | | |
| Sugar beet # | Construct | Empty vector | pthG₁₋₄₈₈ | pthG₆₂₋₂₅₅ | pthG₂₄₅₋₄₈₈ |
| Control | | 100 | 8 | 100 | 100 |
| not transgenic | | | | | |
| PR 171/02 | 4xD-pthG-₆₂₋₂₅₅ -kan | 100 | 7 | n.d. | 32 |
| PR 171/13 | | 100 | 5 | n.d. | 35 |
| PR 171/22 | | 100 | 7 | n.d. | 41 |
| PR 171/16 | | 100 | 1 | n.d. | 41 |
| PR 171/01 | | 100 | 8 | n.d. | 47 |
| PR 171/14 | | 100 | 4 | n.d. | 48 |
| PR 171/09 | | 100 | 5 | n.d. | 53 |
| PR 171/06 | | 100 | 3 | n.d. | 60 |
| PR 171/24 | | 100 | 19 | n.d. | 61 |
| PR 171/19 | | 100 | 2 | n.d. | 64 |
| PR 171/12 | | 100 | 8 | n.d. | 100 |
| PR 173/17 | 4xD-pthG-₂₄₅₋₄₈₈ -kan | 100 | 1 | 18 | n.d. |
| PR 173/15 | | 100 | 3 | 40 | n.d. |
| PR 173/12 | | 100 | 7 | 76 | n.d. |
| PR 173/11 | | 100 | 6 | 87 | n.d. |
| PR 173/05 | | 100 | 8 | 100 | n.d. |

### Combination of the Avirulence Protein, Divided in Two, by Crossing Selected Sugar Beet Transformants

The functionally characterised transgenic lines PR144, PR148, PR171 and PR173 could be used for different crossings (see FIG. 7 and Table 6 by way of example). One possibility lies in the crossing of transgenic sugar beets in which the two pthG partial fragments are under the control of the same pathogen-inducible promoter, for example the 2×S-2×D promoter. A corresponding crossing was performed for the line PR148/56 with the lines PR144/4 and PR144/30.

The surface of the seed obtained from the crossings was disinfected and exposed to tissue culture conditions on MS medium. The in-vitro plants thus obtained were examined by means of PCR for the presence of the two pthG fragments and were clonally multiplied. The PCR analysis showed that crossing numbers 5258/1 and 5260/1, which originate from the crossing PR148/56×PR144/4, and crossing numbers 5398/1 and 5398/3, which are descendants of the crossing PR148/56×PR144/30, bore the sequences pthG₁₋₂₅₅ and pthG₁₂₁₋₄₈₈. The multiplied in-vitro plants were infected in the in-vitro test with C. beticola in accordance with Schmidt et al., 2008. Infected and uninfected plants were harvested 1, 2, 4 and 7 days after inoculation (three biological replicas in each case), RNA was isolated as described, and a qRT-PCR analysis was performed in order to detect the transcription accumulation of pthG₁₋₂₅₅ and pthG₁₂₁₋₄₈₈ after Cercospora beticola infection. Non-transgenic sugar beet plants (3DC4156) and transgenic sugar beet plants, which had been transformed with the construct FP635 under the control of a pathogen-inducible promoter (PR167/11), were used as controls. FP635 codes for a red-fluorescing protein with an excitation at a wavelength maximum of 589 nm and a light emission at a wavelength maximum of 636 nm. It has no effect itself on the plant pathogen defence. The results showed that the nucleic acid pthG₁₋₂₅₅ in the PR148×PR144 crossings is expressed weakly and is induced by the pathogen attack (FIG. 8A). Although the transgenic line PR148/56 in the transient complementation test displayed no cell death, the induction and weak expression of the sequence pthG 1-255 can be detected by sensitive qRT-PCR.

The nucleic acid pthG₁₂₁₋₄₈₈ in the PR148×PR144 crossings was strongly induced as a result of the C. beticola infection (FIG. 8B), whereas no transcription accumulation could be detected in the non-transgenic and transgenic controls. The activities of the promoters of the two nucleic acids thus showed an overlapping expression regulation for the case of a pathogen infection.

TABLE 6
Identification of descendants from the crossings PR148/56 × PR144/4, PR148/56 × PR144/30
and PR144/19 × PR171/19 (kan: kanamycin as selection marker; ¹relative enzyme activity
in the transient complementation test (100% = no cell death, 0% = maximum cell death)

| | Parent 1 | | | Parent 2 | | |
|---|---|---|---|---|---|---|
| Crossing | Promoter and | PCR | Enzyme activity¹ | Promoter and | PCR | Enzyme activity¹ |
| No. | pthG fragment | Parent 1 | Parent 1 [%] | pthG fragment | Parent 2 | Parent 2 [%] |
| 5258/1 | PR148/56 | positive | 100% | PR144/04 | positive | 20% |
| | 2xS-2xD- | | | 2xS-2xD- | | |
| | pthG₁₋₂₅₅ | | | pthG₁₂₁₋₄₈₈ | | |
| 5260/1 | PR148/56 | positive | 100% | PR144/04 | positive | 20% |
| | 2xS-2xD- | | | 2xS-2xD- | | |
| | pthG₁₋₂₅₅ | | | pthG₁₂₁₋₄₈₈ | | |
| 5398/1 | PR148/56 | positive | 100% | PR144/30 | positive | 30% |
| | 2xS-2xD- | | | 2xS-2xD- | | |
| | pthG₁₋₂₅₅ | | | pthG₁₂₁₋₄₈₈ | | |
| 5398/3 | PR148/56 | positive | 100% | PR144/30 | positive | 30% |
| | 2xS-2xD- | | | 2xS-2xD- | | |
| | pthG₁₋₂₅₅ | | | pthG₁₂₁₋₄₈₈ | | |
| PR5021- | PR171/19 | positive | 64% | PR144/19 | positive | 62% |
| 2010-T- | 4xD- | | | 2xS-2xD- | | |
| 003 | pthG₆₂₋₂₅₅ | | | pthG₁₂₁₋₄₈₈ | | |
| PR5022- | PR171/19 | positive | 64% | PR144/19 | positive | 62% |
| 2010-T- | 4xD- | | | 2xS-2xD- | | |
| 002 | pthG₆₂₋₂₅₅ | | | pthG₁₂₁₋₄₈₈ | | |

Detection of the Boosted Pathogen Defence Reaction by Quantification of the Fungal Biomass after Cercospora beticola Infection by Means of Expression Analysis

An improved pathogen defence could be detected by the quantitative determination of pathogen-specific expression transcripts, which constituted a measure for the pathogenic biomass as a result of the pathogen infection. For this purpose, the accumulation of the C. beticola gene for the 60S ribosomal protein (FIG. 9A) was quantified by qRT-PCR with the primers S1420 (SEQ ID NO: 52) and S1421 (SEQ ID NO: 53). Each three biological replicas of the PR148×PR144 crossings 5258/1, 5260/1, 5398/1 and 5398/3 were analysed on day 1, 2, 4 and 7 after Cercospora beticola infection. 3DC4156 and PR167/11 were still used as controls. The result showed that the fungal biomass of the control plants at the end of the test, 7 days after the infection, was approximately 50% higher than that of the transgenic plants of the PR148×PR144 crossings. This result proved that a pathogen defence reaction was triggered in the plants by means of the co-expression of pthG₁₋₂₅₅ and pthG₁₂₁₋₄₈₈ as a result of the C. beticola infection, and this reaction, by contrast with the controls, was able to provoke a delayed and limited spread of the pathogen C. beticola.

### Detection of the Boosted Pathogen Defence Reaction by Quantification of Components of the Plant Pathogen Defence by Means of Expression Analysis

For detection of an increased pathogen response in one of the PR148×PR144 crossings, the expression level of components of the plant pathogen defence are determined quantitatively. For this purpose, the transcript accumulation of the B. vulgaris gene of the caffeic acid o-methyl transferase BvCoMT (FIG. 9B), which is induced in the case of resistance reactions of sugar beet with respect to C. beticola (Weltmeier et al., 2011), was performed in each case in three biological replicates of the PR148×PR144 crossings (in-vitro plants) on day 1, 2, 4 and 7 after Cercospora beticola infection by means of qRT-PCR as described with Weltmeier et al. 2011 for the gene PLT3_{—}005_g06.f (FIG. 9B). The result showed that the expression of the BvCOMT gene, which was expressed in conjunction with the pathogen defence, in the transgenic in-vitro plants of the PR148×PR144 crossings 5398/1 and 5398/3 is higher compared with the controls (3DC4156 and PR167/11) seven days after the C. beticola infection. The expression in the case of the crossing numbers 5258/1 and 5260/1 was comparable to the controls. Since the fungal biomass in the controls, however, was 50% higher, a stronger BvCOMT transcript quantity should also be expected compared with that in the crossing products. The same or even higher BvCOMT transcription quantity in the crossings shows that the synthesis products of the nucleic acids pthG₁₋₂₅₅ and pthG₁₂₁₋₄₈₈, formed in reaction to the C. beticola infection, lead to an increased plant pathogen response.

Production of Crossing Products in which the pthG Nucleic Acids are Under the Control of Different Pathogen-Induced Promoters

In order to ensure that all parts of the avirulence protein PthG are present in synthesised form in a cell exclusively at the moment of an “actual infection” and that the expression thereof is not induced by other stimuli (transformation, development, abiotic stress, etc.), the use of two different synthetic pathogen-inducible promoters is particularly advantageous. For this reason, a crossing of selected PR144 (construct 2×S-2×D-pthG₁₂₁₋₄₈₈) and PR171 (construct 4×D-pthG₆₂₋₂₅₅) transformants was performed on the basis of the functionally analysed transformants (Tables 4 and 5). Seed was obtained from the crossings. PR171/19×PR144/4, PR171/19×PR144/5, PR171/19×PR144/19 and also PR171/2×PR144/4 and PR171/2×PR144/19, however, only the crossing PR171/19×PR144/19 led to viable descendants.

A large number of the obtained crossing products showed a low induction of an HR reaction in spite of the excellent pathogen specificity of the used synthetic promoters, even in the absence of a pathogen above all during the germination phase. This was generally sufficient to allow the sensitive seedlings to die (FIG. 10A and FIG. 10B). However, vigorous sugar beet plants in which an induction of this type was absent during the germination phase were able to be grown successfully from some crossings (FIG. 10C), to root (FIG. 10D) and to be transferred to the greenhouse. There, the plants developed inconspicuously (FIG. 10E).

In view of the functional properties of the selected crossing partners, two conclusions can be drawn. PR171/17 and PR144/19, with 64% and 62% respectively of relative enzyme activity in the transient complementation test, show a lower cell death induction than the other selected crossing partners (Table 7). The inducibility or expression of the partial fragments should not be too strong in the parent lines and on the other hand a suitable combination of parents can ultimately be found by the gradual selection of different activity levels.

An infection of the crossing products PR5021-2010-T-003 (PR171/17×PR144/19) with C. beticola transferred into the greenhouse showed, after qRT-PCR analysis, that the transcription both of the sequence pthG₆₂₋₂₅₅ and also of the sequence pthG₁₂₁₋₄₈₈ are induced strongly in the plants 11 days after inoculation (FIG. 11).

TABLE 7
Results of the crossing of transparent pthG plants with different
functional activity. The percentages in brackets specify the
relative enzyme activity of the transgenic lines in the transient
complementation test (Table 4 and 5).

| | | | | Vigorous |
|---|---|---|---|---|
| Parent 1 | Parent 2 | Crossing | Seed | seedling |
| PR171/19 | PR144/4 | PR171/19 × PR144/4 | yes | no |
| (64%) | (20%) | | | |
| PR171/19 | PR144/5 | PR171/19 × PR144/5 | yes | no |
| (64%) | (55%) | | | |
| PR171/19 | PR144/19 | PR171/19 × PR144/19 | yes | yes |
| (64%) | (62%) | | | |
| PR171/2 | PR144/4 | PR171/2 × PR144/4 | yes | no |
| (32%) | (20%) | | | |
| PR171/2 | PR144/19 | PR171/2 × PR144/19 | yes | no |
| (32%) | (62%) | | | |

### REFERENCES

Altschul, S. F., Gish, W., Miller, W., Myers, E. W. und Lipman, D. J. (1990) Basic local alignment search tool. Journal of Molecular Biology 215(3): 403-410.
Belbahri, L., Boucher, C., Candresse, T., Nicole, M., Ricci, P. und Keller, H. (2001) A local accumulation of the Ralstonia solanacearum PopA protein in transgenic tobacco renders a compatible plant-pathogen interaction incompatible. The Plant Journal 28(4): 419-430.
Dangl, J. L. und Jones, J. D. G. (2001). Plant pathogens and integrated defence responses to infection. Nature 411: 826-833.
de Wit, P. J. G. M. (1992) Molecular characterization of gene-for-gene systems in plant-fungus interactions and the application of avirulence genes in control of plant pathogens. Annual Review of Phytopathology 30: 391-418.
Ezra, D., Barash, I., Valinky, L. und Manulis, S. (2000) The dual function in virulence and host range restriction of a gene isolated from the pPATH_{Ehg} plasmid of Erwinia herbicola pv. gypsophilae. Molecular Plant-Microbe Interactions 13(6), 683-692.
Ezra, D., Barash, I., Weinthal, D. M., Gaba, V. und Manulis, S. (2004) pthG from Pantoea agglomerans pv. gypsophilae encodes an avirulence effector that determines incompatibility in multiple beet species. Molecular Plant Pathology 5(2), 105-113.
Flor, H. H. (1971) Current status of the gene-for-gene concept. Annual Review of Phytopathology 9: 275-296.
Gürel, S., Gûrel, E. and Kaya, Z. (2000). Doubled haploid plant production from unpollinated ovules of sugar beet (Beta vulgaris L.). Plant Cell Reports 19: 1155-1159.
Gutierrez, J. R., Balmuth, A. L., Ntoukakis, V., Mucyn, T. S., Gimenez-Ibanez, S., Jones, A. M. E. und Rathjen, J. P. (2010) Prf immune complexes of tomato are oligomeric and contain multiple Pto-like kinases that diversify effector recognition. The Plant Journal 61, 507-518.
Jones, J. D. G. und Dangl, J. L. (2006) The plant immune system. Nature 444, 323-329
Joosten, M. H. A. J. und de Wit, P. J. G. M. (1999) The tomato-Cladosporium fulvum interaction: A versatile experimental system to study plant-pathogen interactions. Annual Review of Phytopathology 37: 335-367.
Keller, H., Pamboukdjian, N., Ponchet, M., Poupet, A., Delon, R., Verrier, J.-L., Roby, D., and Ricci, P., 1999: Pathogen-induced elicitin production in transgenic tobacco generates a hypersensitive response and nonspecific disease resistance. Plant Cell 11: 223-235.
Kuta, D. D. und Tripathi, L. (2005) Agrobacterium-induced hypersensitive necrotic reaction in plant cells: a resistance response against Agrobacterium-mediated DNA transfer. African Journal of Biotechnology 4(8): 752-757.
Lindsey, K. und Gallois, P. (1990) Transformation of Sugarbeet (Beta vulgaris) by Agrobacterium tumefaciens. Journal of Experimental Botany 226(41): 529-536.
Martini, N., Egen, M., Rüntz, I., und Strittmatter, G., 1993: Promoter sequences of a potato pathogenesis-related gene mediate transcriptional activation selectively upon fungal infection. Molecular and General Genetics 236: 179-186.
Rushton, P. J., Reinstadler, A., Lipka, V., Lippok, B. und Somssich, I. E., 2002: Synthetic plant promoters containing defined regulatory elements provide novel insights into pathogen- and wound-induced signaling. Plant Cell 14(4), 749-62.
Sambrook, J., Fritsch, E. F. und Maniatis, T. (1989) Molecular Cloning. A laboratory manual. Cold Spring Harbor Laboratory Press, 2. Aufl.
Schmidt, K., Heberle, B., Kurrasch J., Nehls, R. and Stahl D. J. (2004) Suppression of phenylalanine ammonia lyase expression in sugar beet by the fungal pathogen Cercospora beticola is mediated at the core promoter of the gene. Plant Molecular Biology 55: 835-852.
Schmidt, K., Pflugmacher, M., Klages, S., Mäser, Mock, A. und Stahl D. J. (2008) Accumulation of the hormone abscisic acid (ABA) at the infection site of the fungus Cercospora beticola supports the role of ABA as a repressor of plant defence in sugar beet. Molecular Plant Pathogology 9(5): 661-673.
Stahl, D. J., Kloos, D. U., and Hehl, R. (2004). A sugar beet chlorophyll a/b binding protein void of G-box like elements confer strong and leaf specific reporter gene expression in transgenic sugar beet. BMC Biotechnology 4; 31: 12
Stahl, D. J., Schmidt, K. und Nehls, R. (2006) Einsatz gentechnischer Methoden zur Verbesserung der Kulturpflanzenresistenz gegenûber parasitaren Pilzen. Schriftenreihe der Deutschen Phytomedizinischen Gesellschaft e.V. Bd. 8: 80-93.
Strittmatter, G., Gheysen, G., Gianinazzi-Pearson, V., Hahn, K., Niebel, A., et al. (1996) Infections with various types of organisms stimulate transcription from a short promoter fragment of the potato gst1 gene. Molecular Plant-Microbe Interactions 9:68-73.
Weltmeier, F., Maser, A., Menze, A., Hennig, S., Schad, M., Breuer, F., Schulz, B., Holtschulte, B., Nehls, R., and Stahl, D. J. (2011). Transcript profiles in sugar beet genotypes uncover timing and strength of defense reactions to Cercospora beticola infection. Mol. Plant Microbe Interat. 24, 758-772.
WO/1991/15585 (Rijkslandbouwhogeschool Wageningen). Method for protection of plants against pathogens.
WO/1995/31564 (Gatsby Charitable Foundation). Method of introducing pathogen resistance in plants.
WO/1999/43823 (Pioneer Hi-Bred International, Inc.). Methods for enhancing disease resistance in plants.
WO/2000/29592 (Max-Planck-Gesellschaft zur Förderung der Wissenschaften e.V.). Chimeric promoters capable of mediating gene expression in plants upon pathogen infection and uses thereof
WO/2006/128444 (KWS SAAT AG). Autoaktiviertes Resistenzprotein.
WO/2007/068935 (Plant Bioscience Ltd.). Methods, means and compositions for enhancing Agrobacterium-mediated plant cell transformation efficiency.
WO/2007/147395 (KWS SAAT AG). Pathogen induzierbarer synthetischer Promotor.

1. A pathogen-resistant plant comprising at least two nucleic acids integrated into the genome in a stable manner, wherein the nucleic acids
(i) code for different parts of an avirulence protein and
(ii) are operatively linked to promoters,
wherein at least one of the promoters is pathogen-inducible.

2. The plant according to claim 1, wherein the different parts of the avirulence protein individually do not induce a plant pathogen defence reaction.

3. The plant according to claim 1, wherein the different parts of the avirulence protein have an amino acid sequence that is identical or similar in portions.

4. The plant according to claim 3, wherein the amino acid sequence that is identical or similar in portions has a length of more than 3 successive amino acids.

5. The plant according to claim 1, wherein a resistance gene to the avirulance protein is contained naturally in the genome of the plant.

6. The plant according to claim 1, wherein the operatively linked promoters each have such a specificity wherein the different parts of the avirulence protein are expressed in a manner delimited locally to the pathogen infection site of the plant.

7. The plant according to claim 1, wherein the plant is of the genus Beta.

8. The plant according to claim 7, wherein the avirulence protein is coded by an avirulence gene selected from the group consisting of:
(i) SEQ ID NO: 1 or SEQ ID NO: 3,
(ii) a nucleotide sequence that codes for an amino acid sequence SEQ ID NO: 2 or SEQ ID NO: 4,
(iii) a nucleotide sequence complementary to the nucleotide sequence from (i) or (ii),
(iv) a nucleotide sequence that hybridises with one of the nucleotide sequences from (i), (ii) or (iii) under stringent conditions,
(v) a nucleotide sequence with a homology of at least 90% at DNA level to one of the nucleotide sequences from (i), (ii) or (iii), and
(vi) a nucleotide sequence that codes for an amino acid sequence having an identity of at least 90% or a similarity of at least 90% compared with the amino acid sequence SEQ ID NO: 2.

9. The plant according to claim 7, wherein one of the at least two nucleic acids comprises a nucleotide sequence selected from the group consisting of:
(i) SEQ ID NO: 35,
(ii) a fragment of SEQ ID NO: 35, comprising SEQ ID NO: 54,
(iii) SEQ ID NO: 5, SEQ ID NO: 7, SEQ ID NO: 9, SEQ ID NO: 29, SEQ ID NO: 31 or SEQ ID NO: 33,
(iv) a nucleotide sequence that codes for an amino acid sequence SEQ ID NO: 36,
(v) a nucleotide sequence that codes for a fragment of the amino acid sequence SEQ ID NO: 36, comprising an amino acid sequence SEQ ID NO: 55,
(vi) a nucleotide sequence that codes for an amino acid sequence SEQ ID NO: 6, SEQ ID NO: 8, SEQ ID NO: 10, SEQ ID NO: 30, SEQ ID NO: 32 or SEQ ID NO: 34,
(vii) a nucleotide sequence complementary to one of the nucleotide sequences from (i) to (vi),
(viii) a nucleotide sequence that hybridises with one of the nucleotide sequences from (i) to (vii) under stringent conditions,
(ix) a nucleotide sequence with a homology of at least 90% at DNA level to one of the nucleotide sequences from (i) to (vii), and
(x) a nucleotide sequence that codes for an amino acid sequence having an identity of at least 90% or a similarity of at least 90% compared with one of the amino acid sequences SEQ ID NO: 36, SEQ ID NO: 6, SEQ ID NO: 8, SEQ ID NO: 10, SEQ ID NO: 30, SEQ ID NO: 32 or SEQ ID NO: 34 or with a fragment of the amino acid sequence SEQ ID NO: 36, comprising an amino acid sequence SEQ ID NO: 55.

10. The plant according to claim 7, wherein one of the at least two nucleic acids comprises a nucleotide sequence selected from the group consisting of:
(i) SEQ ID NO: 13,
(ii) a fragment of SEQ ID NO: 13, comprising SEQ ID NO: 56,
(iii) SEQ ID NO: 17, SEQ ID NO: 19, SEQ ID NO: 21, SEQ ID NO: 23, SEQ ID NO: 25 or SEQ ID NO: 27,
(iv) a nucleotide sequence that codes for an amino acid sequence SEQ ID NO: 14,
(v) a nucleotide sequence that codes for a fragment of the amino acid sequence SEQ ID NO: 14, comprising an amino acid sequence SEQ ID NO: 57,
(vi) a nucleotide sequence that codes for an amino acid sequence SEQ ID NO: 18, SEQ ID NO: 20, SEQ ID NO: 22, SEQ ID NO: 24, SEQ ID NO: 26 or SEQ ID NO: 28,
(vii) a nucleotide sequence complementary to one of the nucleotide sequences from (i) to (vi),
(viii) a nucleotide sequence that hybridises with one of the nucleotide sequences from (i) to (vii) under stringent conditions,
(ix) a nucleotide sequence with a homology of at least 90% at DNA level to one of the nucleotide sequences from (i) to (vii), and
(x) a nucleotide sequence that codes for an amino acid sequence having an identity of at least 90% or a similarity of at least 90% compared with one of the amino acid sequences SEQ ID NO: 14, SEQ ID NO: 18, SEQ ID NO: 20, SEQ ID NO: 22, SEQ ID NO: 24, SEQ ID NO: 26 or SEQ ID NO: 28 or with a fragment of the amino acid sequence SEQ ID NO: 14, comprising an amino acid sequence SEQ ID NO: 57.

11. A seed, part, organ, tissue or cell of the plant according to claim 1.

12. A composition comprising at least two nucleic acids, wherein the nucleic acids
(i) code for different parts of an avirulence protein and
(ii) are operatively linked to promoters,
and at least one of the promoters is pathogen-inducible.

13. The composition according to claim 12, wherein one of the at least two nucleic acids comprises a nucleotide sequence selected from the the group consisting of:
(i) SEQ ID NO: 35,
(ii) a fragment of SEQ ID NO: 35, comprising SEQ ID NO: 54,
(iii) SEQ ID NO: 5, SEQ ID NO: 7, SEQ ID NO: 9, SEQ ID NO: 29, SEQ ID NO: 31 or SEQ ID NO: 33,
(iv) a nucleotide sequence that codes for an amino acid sequence SEQ ID NO: 36,
(v) a nucleotide sequence that codes for a fragment of the amino acid sequence SEQ ID NO: 36, comprising an amino acid sequence SEQ ID NO: 55,
(vi) a nucleotide sequence that codes for an amino acid sequence SEQ ID NO: 6, SEQ ID NO: 8, SEQ ID NO: 10, SEQ ID NO: 30, SEQ ID NO: 32 or SEQ ID NO: 34,
(vii) a nucleotide sequence complementary to one of the nucleotide sequences from (i) to (vi),
(viii) a nucleotide sequence that hybridises with one of the nucleotide sequences from (i) to (vii) under stringent conditions,
(ix) a nucleotide sequence with a homology of at least 90% at DNA level to one of the nucleotide sequences from (i) to (vii), and
(x) a nucleotide sequence that codes for an amino acid sequence having an identity of at least 90% or a similarity of at least 90% compared with one of the amino acid sequences SEQ ID NO: 36, SEQ ID NO: 6, SEQ ID NO: 8, SEQ ID NO: 10, SEQ ID NO: 30, SEQ ID NO: 32 or SEQ ID NO: 34 or with a fragment of the amino acid sequence SEQ ID NO: 36, comprising an amino acid sequence SEQ ID NO: 55.

14. The composition according to claim 12, wherein one of the at least two nucleic acids comprises a nucleotide sequence selected from the following group:
(i) SEQ ID NO: 13,
(ii) a fragment of SEQ ID NO: 13, comprising SEQ ID NO: 56,
(iii) SEQ ID NO: 17, SEQ ID NO: 19, SEQ ID NO: 21, SEQ ID NO: 23, SEQ ID NO: 25 or SEQ ID NO: 27,
(iv) a nucleotide sequence that codes for an amino acid sequence SEQ ID NO: 14,
(v) a nucleotide sequence that codes for a fragment of the amino acid sequence SEQ ID NO: 14, comprising an amino acid sequence SEQ ID NO: 57,
(vi) a nucleotide sequence that codes for an amino acid sequence SEQ ID NO: 18, SEQ ID NO: 20, SEQ ID NO: 22, SEQ ID NO: 24, SEQ ID NO: 26 or SEQ ID NO: 28,
(vii) a nucleotide sequence complementary to one of the nucleotide sequences from (i) to (vi),
(viii) a nucleotide sequence that hybridises with one of the nucleotide sequences from (i) to (vii) under stringent conditions,
(ix) a nucleotide sequence with a homology of at least 90% at DNA level to one of the nucleotide sequences from (i) to (vii), and
(x) a nucleotide sequence that codes for an amino acid sequence having an identity of at least 90% or a similarity of at least 90% compared with one of the amino acid sequences SEQ ID NO: 14, SEQ ID NO: 18, SEQ ID NO: 20, SEQ ID NO: 22, SEQ ID NO: 24, SEQ ID NO: 26 or SEQ ID NO: 28 or with a fragment of the amino acid sequence SEQ ID NO: 14, comprising an amino acid sequence SEQ ID NO: 57.

15. Parent plants for producing a plant according to claim 1, wherein the parent plant has been transformed in a stable manner with at least one, but not with all, of the nucleic acids.

16. A seed, part, organ, tissue or cell of the parent plants according to claim 15.

17. A method for producing a plant according to claim 15.

18. The plant according to claim 4, wherein the amino acid sequence that is identical or similar in portions has a length of at least 11 successive amino acids.

19. The plant according to claim 1, wherein a resistance gene to the avirulance protein has been inserted via gene engineering or breeding methods.

20. A method for producing a plant according to claim 1.

21. A recombinant vector which comprises at least one of the operatively linked nucleic acids of claim 1.

22. The plant according to claim 6, wherein at least one of the promoters is pathogen-inducible and wherein the promoters that are operatively linked to the remaining nucleic acids are induced spatially, temporally, overlapping, or combinations thereof with that of the pathogen-inducible promoter.

23. The plant according to claim 22, wherein the promoters that are operatively linked to the remaining nucleic acids are constitutive, tissue-specific, organ-specific, storage-induced, development-specific or also pathogen-inducible promoters.

24. The plant according to claim 1, wherein the intensity of the pathogen defence reaction can be controlled by the specific combination of the stably integrated nucleic acids.


SEQUENCE LISTING (57 sequences)

SEQ ID NO: 1 - DNA, 1467 bp, Erwinia herbicola
atgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatag

SEQ ID NO: 2 - protein, 488 aa, Erwinia herbicola
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 3 - DNA, 1479 bp, Artificial Sequence
  Description of Artificial Sequence Synthetic pthG_1-488 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 4 - protein, 488 aa, Artificial Sequence; identical to SEQ ID NO: 2
  Description of Artificial Sequence Synthetic PthG_1-488 polypeptide

SEQ ID NO: 5 - DNA, 780 bp, Artificial Sequence
  Description of Artificial Sequence Synthetic pthG_1-255 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgtagcgatggatcc

SEQ ID NO: 6 - protein, 255 aa, Artificial Sequence
  Description of Artificial Sequence Synthetic PthG_1-255 polypeptide
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENL

SEQ ID NO: 7 - DNA, 600 bp, Artificial Sequence
  Description of Artificial Sequence Synthetic pthG_62-255 polynucleotide
ccatggctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgtagcgatggatcc

SEQ ID NO: 8 - protein, 195 aa, Artificial Sequence
  Description of Artificial Sequence Synthetic PthG_62-255 polypeptide
MAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENL

SEQ ID NO: 9 - DNA, 510 bp, Artificial Sequence
  Description of Artificial Sequence Synthetic pthG_92-255 polynucleotide
ccatggcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgtagcgatggatcc

SEQ ID NO: 10 - protein, 165 aa, Artificial Sequence
  Description of Artificial Sequence Synthetic PthG_92-255 polypeptide
MAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENL

SEQ ID NO: 11 - DNA, 423 bp, Artificial Sequence
  Description of Artificial Sequence Synthetic pthG_121-255 polynucleotide
ccatggcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgtagcgatggatcc

SEQ ID NO: 12 - protein, 136 aa, Artificial Sequence
  Description of Artificial Sequence Synthetic PthG_121-255 polypeptide
MASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENL

SEQ ID NO: 13 - DNA, 1122 bp, Artificial Sequence
  Description of Artificial Sequence Synthetic pthG_121-488 polynucleotide
ccatggcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 14 - protein, 369 aa, Artificial Sequence
  Description of Artificial Sequence Synthetic PthG_121-488 polypeptide
MASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 15 - DNA, 1209 bp, Artificial Sequence
  Description of Artificial Sequence Synthetic pthG_92-488 polynucleotide
ccatggcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 16 - protein, 398 aa, Artificial Sequence
  Description of Artificial Sequence Synthetic PthG_92-488 polypeptide
MAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 17 - DNA, 999 bp, Artificial Sequence
  Description of Artificial Sequence Synthetic pthG_162-488 polynucleotide
ccatgggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 18 - protein, 328 aa, Artificial Sequence
  Description of Artificial Sequence Synthetic PthG_162-488 polypeptide
MGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 19 - DNA, 870 bp, Artificial Sequence
  Description of Artificial Sequence Synthetic pthG_205-488 polynucleotide
ccatgggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 20 - protein, 285 aa, Artificial Sequence
  Description of Artificial Sequence Synthetic PthG_205-488 polypeptide
MGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 21 - DNA, 750 bp, Artificial Sequence
  Description of Artificial Sequence Synthetic pthG_245-488 polynucleotide
ccatgggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 22 - protein, 245 aa, Artificial Sequence
  Description of Artificial Sequence Synthetic PthG_245-488 polypeptide
MGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 23 - DNA, 726 bp, Artificial Sequence
  Description of Artificial Sequence Synthetic pthG_253-488 polynucleotide
ccatggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 24 - protein, 237 aa, Artificial Sequence
  Description of Artificial Sequence Synthetic PthG_253-488 polypeptide
MENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 25 - DNA, 720 bp, Artificial Sequence
  Description of Artificial Sequence Synthetic pthG_G+256-488 polynucleotide
ccatgggccacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 26 - protein, 235 aa, Artificial Sequence
  Description of Artificial Sequence Synthetic PthG_G+256-488 polypeptide
MGHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 27 - DNA, 714 bp, Artificial Sequence
  Description of Artificial Sequence Synthetic pthG_257-488 polynucleotide
ccatggacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgccatgcatagcgatggatcc

SEQ ID NO: 28 - protein, 233 aa, Artificial Sequence
  Description of Artificial Sequence Synthetic PthG_257-488 polypeptide
MDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHCHA

SEQ ID NO: 29 - DNA, 1065 bp, Artificial Sequence
  Description of Artificial Sequence Synthetic pthG_1-350 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggtagcgatggatcc

SEQ ID NO: 30 - protein, 350 aa, Artificial Sequence
  Description of Artificial Sequence Synthetic PthG_1-350 polypeptide
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSR

SEQ ID NO: 31 - DNA, 1155 bp, Artificial Sequence
  Description of Artificial Sequence Synthetic pthG_1-380 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtagcgatggatcc

SEQ ID NO: 32 - protein, 380 aa, Artificial Sequence
  Description of Artificial Sequence Synthetic PthG_1-380 polypeptide
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWA

SEQ ID NO: 33 - DNA, 1251 bp, Artificial Sequence
  Description of Artificial Sequence Synthetic pthG_1-412 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggcctagcgatggatcc

SEQ ID NO: 34 - protein, 412 aa, Artificial Sequence
  Description of Artificial Sequence Synthetic PthG_1-412 polypeptide
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEA

SEQ ID NO: 35 - DNA, 1335 bp, Artificial Sequence
  Description of Artificial Sequence Synthetic pthG_1-440 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgctagcgatggatcc

SEQ ID NO: 36 - protein, 440 aa, Artificial Sequence
  Description of Artificial Sequence Synthetic PthG_1-440 polypeptide
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGC

SEQ ID NO: 37 - DNA, 1473 bp, Artificial Sequence
  Description of Artificial Sequence Synthetic pthG_1-486 polynucleotide
ccatgggatgtttcaacgttacgggtgccagtggcagagcgaacaattatgttgtcgagcaccacgacgatcggagttcgatagggcaggaaagccccaggcaagagattgctgatagtccgctgagtcgatcaggccgttcatccaatagaacctctccccgcgtatcgctggacaggctcagagctttcaatcccccaagcggcgcgtcagttggcgctttgagaaattggttctggcaaggacaggctgctcaacatctgcagcagctcgaagcagagctcaacaacggttcgaacattcgcgagctgttggacggccctggatttggcggattgctgacccatgtgatcgacgatatcgcctcgatttcaaacgcggaaaatttgtcgccgatcgaggtcgcagaggagctaaggcaagctattagcaccgggcgcggggcgaacaatttggatatatctttgaaaaacgttctacaattcggactttatgcggtcagggaaaaactgcaaggggaagccgagctcgccggctcgatgcaaacacaccctgcttttcagtctttcccggtggcccaggcctggaaattttgcattgagcaagagcattggggtgattacggcgccgcatccggcatgcgatttgacaacgagcccggctacatgggtagtatgtttcgcggcttaaaattcgtcatcgactcggaagcgggttcctcgtcccgaaaaaaaggcgaaatcgatgctacttggttggagaatttgcacgacatcgctgtcaatggcgtgttccagcgcagagtcatgcatgattggcagttcggcgccaatgtcgaagaagctgtgaatgcagtcgccgcccttaccgcgcctggcagcaatattagcgataaggtcaggagcgcagcgtcggcaatcttctatttgccggaacacctatggccattggagggtgggtatcgacaaggccagttggtccagttcaaactggtttatcccaagaatctgactccggctgggctcggcgatctgcaaaagttccagtcccggttcgccgggtgggctgagtttcgaagtgtcggtaatcgtccgatcgactgggatgaggtagaacgcggcaacgcccaggttatgtgggcgtgcacgggaaaaagccgaaaagaggttatgaagctggtgaaaaatgtgctccaaacgcattataacgatataaaaaggactaccaacgcagaggccgcactgcatgtggtggcacgaacgtgcgctaccctggagcgcgcgcacgtgttcatggatggcagcgcacgaactttcggttgcctgctcctcaataaacttttgctttccgcaggcttatcaccatgcatggcccctgatgccaacgaattcgacggttactcgaacgaagaactggtggcgcggattcaggaaggacagaatatattccgggcacattgctagcgatggatcc

SEQ ID NO: 38 - protein, 486 aa, Artificial Sequence
  Description of Artificial Sequence Synthetic PthG_1-486 polypeptide
MGCFNVTGASGRANNYVVEHHDDRSSIGQESPRQEIADSPLSRSGRSSNRTSPRVSLDRLRAFNPPSGASVGALRNWFWQGQAAQHLQQLEAELNNGSNIRELLDGPGFGGLLTHVIDDIASISNAENLSPIEVAEELRQAISTGRGANNLDISLKNVLQFGLYAVREKLQGEAELAGSMQTHPAFQSFPVAQAWKFCIEQEHWGDYGAASGMRFDNEPGYMGSMFRGLKFVIDSEAGSSSRKKGEIDATWLENLHDIAVNGVFQRRVMHDWQFGANVEEAVNAVAALTAPGSNISDKVRSAASAIFYLPEHLWPLEGGYRQGQLVQFKLVYPKNLTPAGLGDLQKFQSRFAGWAEFRSVGNRPIDWDEVERGNAQVMWACTGKSRKEVMKLVKNVLQTHYNDIKRTTNAEAALHVVARTCATLERAHVFMDGSARTFGCLLLNKLLLSAGLSPCMAPDANEFDGYSNEELVARIQEGQNIFRAHC

SEQ ID NO: 39 - DNA, 35 bp, Artificial Sequence
  Description of Artificial Sequence Synthetic primer S549
aaccatggctttcaatcccccaagcggcgcgtcag

SEQ ID NO: 40 - DNA, 33 bp, Artificial Sequence
  Description of Artificial Sequence Synthetic primer S544
cgggcacattgccatgcatagcgatggatccaa

SEQ ID NO: 41 - DNA, 33 bp, Artificial Sequence; identical to SEQ ID NO: 40
  Description of Artificial Sequence Synthetic primer S558

SEQ ID NO: 42 - DNA, 34 bp, Artificial Sequence
  Description of Artificial Sequence Synthetic primer S550
aaccatggcctcgatttcaaacgcggaaaatttg

SEQ ID NO: 43 - DNA, 34 bp, Artificial Sequence
  Description of Artificial Sequence Synthetic primer S551
aaccatggacatcgctgtcaatggcgtgttccag

SEQ ID NO: 44 - DNA, 36 bp, Artificial Sequence
  Description of Artificial Sequence Synthetic primer S545
agaactaagccatgggatgtttcaacgttacgggtg

SEQ ID NO: 45 - DNA, 42 bp, Artificial Sequence
  Description of Artificial Sequence Synthetic primer S552
aaggatccatcgctagcaatgtgcccggaatatattctgtcc

SEQ ID NO: 46 - DNA, 35 bp, Artificial Sequence
  Description of Artificial Sequence Synthetic primer S561
ttggatccatcgctagcaaccgaaagttcgtgcgc

SEQ ID NO: 47 - DNA, 45 bp, Artificial Sequence
  Description of Artificial Sequence Synthetic primer S560
ttggatccatcgctaggcctctgcgttggtagtcctttttatatc

SEQ ID NO: 48 - DNA, 33 bp, Artificial Sequence
  Description of Artificial Sequence Synthetic primer S559
ttggatccatcgctacgcccacataacctgggc

SEQ ID NO: 49 - DNA, 40 bp, Artificial Sequence
  Description of Artificial Sequence Synthetic primer S553
ttggatccatcgctaccgggactggaacttttgcagatcg

SEQ ID NO: 50 - DNA, 38 bp, Artificial Sequence
  Description of Artificial Sequence Synthetic primer S562
ttggatccatcgctagcctttttttcgggacgaggaac

SEQ ID NO: 51 - DNA, 37 bp, Artificial Sequence
  Description of Artificial Sequence Synthetic primer S554
ggatccatcgctacaaattctccaaccaagtagcatc

SEQ ID NO: 52 - DNA, 21 bp, Artificial Sequence
  Description of Artificial Sequence Synthetic primer S1420
gaggccatccacgagctcaag

SEQ ID NO: 53 - DNA, 22 bp, Artificial Sequence
  Description of Artificial Sequence Synthetic primer S1421
agcccatgtcatcgtcctcctc

SEQ ID NO: 54 - DNA, 87 bp, Artificial Sequence
  Description of Artificial Sequence Synthetic pthG_92-120 oligonuleotide
gcagagctcaacaacggttcgaacattcgcgagctgttggacggccctggatttggcggattgctgacccatgtgatcgacgatatc

SEQ ID NO: 55 - protein, 29 aa, Artificial Sequence
  Description of Artificial Sequence Synthetic PthG_92-120 peptide
AELNNGSNIRELLDGPGFGGLLTHVIDDI

SEQ ID NO: 56 - DNA, 138 bp, Artificial Sequence
  Description of Artificial Sequence Synthetic pthG_441-486 polynucleotide
ctgctcctcaataaacttttgctttccgcaggcttatcaccatgcatggcccctgatgccaacgaattcgacggttactcgaacgaagaactggtggcgcggattcaggaaggacagaatatattccgggcacattgc

SEQ ID NO: 57 - protein, 46 aa, Artificial Sequence
  Description of Artificial Sequence Synthetic PthG_441-486 polypeptide
LLLNKLLLSAGLSPCMAPDANEFDGYSNEELVARIQEGQNIFRAHC